SEQUENCE LISTING (69 sequences)

SEQ ID NO: 1 - protein, 702 aa, synthetic construct
MEFGLSWVFLVALFRGVQSQVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 691 aa, synthetic construct
MEFGLSWVFLVALFRGVQSQVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 3 - protein, 677 aa, synthetic construct
MEFGLSWVFLVALFRGVQSQVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 4 - protein, 507 aa, synthetic construct
MEFGLSWVFLVALFRGVQSQVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSILMQYIKANSKFIGIPMGLPQSIALSSLMVAQ

SEQ ID NO: 5 - protein, 488 aa, synthetic construct
MEFGLSWVFLVALFRGVQSQVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSAKFVAAWTLKAAA

SEQ ID NO: 6 - protein, 733 aa, synthetic construct
MEFGLSWVFLVALFRGVQSQVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSGGGGSGGGGSILMQYIKANSKFIGIPMGLPQSIALSSLMVAQ

SEQ ID NO: 7 - protein, 466 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 8 - protein, 455 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 9 - protein, 271 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSILMQYIKANSKFIGIPMGLPQSIALSSLMVAQ

SEQ ID NO: 10 - protein, 252 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAA

SEQ ID NO: 11 - protein, 289 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAAPMGLPILMQYIKANSKFIGIPMGLPQSIALSSLMVAQ

SEQ ID NO: 12 - protein, 497 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSGGGGSGGGGSILMQYIKANSKFIGIPMGLPQSIALSSLMVAQ

SEQ ID NO: 13 - protein, 438 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSTNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPK

SEQ ID NO: 14 - protein, 473 aa, synthetic construct
MEFGLSWVFLVALFRGVQSDIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSTNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKGGSILMQYIKANSKFIGIPMGLPQSIALSSLMVAQ

SEQ ID NO: 15 - protein, 466 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 16 - protein, 455 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 17 - protein, 271 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSILMQYIKANSKFIGIPMGLPQSIALSSLMVAQ

SEQ ID NO: 18 - protein, 252 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAA

SEQ ID NO: 19 - protein, 289 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAAPMGLPILMQYIKANSKFIGIPMGLPQSIALSSLMVAQ

SEQ ID NO: 20 - protein, 497 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSGGGGSGGGGSILMQYIKANSKFIGIPMGLPQSIALSSLMVAQ

SEQ ID NO: 21 - protein, 438 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSTNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPK

SEQ ID NO: 22 - protein, 473 aa, synthetic construct
MEFGLSWVFLVALFRGVQSDIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSTNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKGGSILMQYIKANSKFIGIPMGLPQSIALSSLMVAQ

SEQ ID NO: 23 - protein, 466 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 24 - protein, 455 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 25 - protein, 271 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSILMQYIKANSKFIGIPMGLPQSIALSSLMVAQ

SEQ ID NO: 26 - protein, 252 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAA

SEQ ID NO: 27 - protein, 289 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAAPMGLPILMQYIKANSKFIGIPMGLPQSIALSSLMVAQ

SEQ ID NO: 28 - protein, 497 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSGGGGSGGGGSILMQYIKANSKFIGIPMGLPQSIALSSLMVAQ

SEQ ID NO: 29 - protein, 473 aa, synthetic construct
MEFGLSWVFLVALFRGVQSDIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSTNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKGGSILMQYIKANSKFIGIPMGLPQSIALSSLMVAQ

SEQ ID NO: 30 - protein, 473 aa, synthetic construct; identical to SEQ ID NO: 29

SEQ ID NO: 31 - protein, 470 aa, synthetic construct
MALLVLFLSLAAFPSCGVLSQVQLKESGPGLVAPSQSLSITCTVSGFSLTSYGVHWVRQPPGKGLEWLGVIWAGGSINYNSALMSRLSISKDNFKSQVFLKMSSLQTDDTAMYYCARAYGDYVHYAMDYWGQGTSVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 236 aa, synthetic construct
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 120 aa, synthetic construct
QVTLKESGPVLVKPTETLTLTCTVSGFSLTSYGVHWIRQPPGKALEWLAVIWAGGSISYSTSLMSRLTISKDTSKSQVVLTMTNMDPVDTATYYCARAYGDYVHYAMDYWGQGTLVTVSS

SEQ ID NO: 34 - protein, 107 aa, synthetic construct
DIQMTQSPSSLSASVGDRVTITCRASQEISGYLTWLQQKPGKAPKLLIYAASTLDSGVPSRFSGSRSGTDFTLTISSLQPEDFATYYCLQYTNYPLTFGQGTKLEIK

SEQ ID NO: 35 - protein, 107 aa, synthetic construct
DIQMTQSPSSLSASVGDRVTITCRASQEISGYLTWLQQKPGKAPKLLIYAASTLDSGVPKRFSGSGSGTDFTLTISSLQPEDFATYYCLQYTNYPLTFGQGTKLEIK

SEQ ID NO: 36 - protein, 107 aa, synthetic construct
DIQMTQSPSSLSASVGDRVTITCRASQEISGYLTWLQQKPGKAPKLLIYAASTLDSGVPKRFSGSRSGTDFTLTISSLQPEDFATYYCLQYTNYPLTFGQGTKLEIK

SEQ ID NO: 37 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 38 - protein, 212 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 39 - protein, 198 aa, synthetic construct
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKS

SEQ ID NO: 40 - protein, 212 aa, synthetic construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKS

SEQ ID NO: 41 - protein, 195 aa, synthetic construct
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPK

SEQ ID NO: 42 - protein, 120 aa, synthetic construct
QVQLQESGPGLVKPSETLSLTCTVSGFSLTSYGWSWIRQPPGKGLEWIGYIWAGGSINYNPSLKSRVTISVDTSKNQFSLKLSSVTAADTAVYYCARAYGDYVHYAMDYWGQGTLVTVSS

SEQ ID NO: 43 - protein, 120 aa, synthetic construct
QVTLKESGPVLVKPTETLTLTCTVSGFSLTSYGVSWIRQPPGKALEWLAHIWAGGSISYSTSLKSRLTISKDTSKSQVVLTMTNMDPVDTATYYCARAYGDYVHYAMDYWGQGTLVTVSS

SEQ ID NO: 44 - protein, 120 aa, synthetic construct
QVQLQESGPGLVKPSGTLSLTCAVSGFSLTSYGWSWVRQPPGKGLEWIGEIWAGGSINYNPSLKSRVTISVDKSKNQFSLKLSSVTAADTAVYYCARAYGDYVHYAMDYWGQGTLVTVSS

SEQ ID NO: 45 - protein, 107 aa, synthetic construct
DIQMTQSPSSLSASVGDRVTITCRASQEISGYLAWFQQKPGKAPKSLIYAASSLQSGVPSRFSGSGSGTDFTLTISSLQPEDFATYYCLQYTNYPLTFGQGTKLEIK

SEQ ID NO: 46 - protein, 107 aa, synthetic construct
DIQMTQSPSSLSASVGDRVTITCRASQEISGYLNWYQQKPGKAPKLLIYAASSLQSGVPSRFSGSGSGTDFTLTISSLQPEDFATYYCLQYTNYPLTFGQGTKLEIK

SEQ ID NO: 47 - protein, 107 aa, synthetic construct
AIQMTQSPSSLSASVGDRVTITCRASQEISGYLGWYQQKPGKAPKLLIYAASSLQSGVPSRFSGSGSGTDFTLTISSLQPEDFATYYCLQYTNYPLTFGQGTKLEIK

SEQ ID NO: 48 - protein, 120 aa, synthetic construct
QVQLQESGPGLVKPSETLSLTCTVSGFSLTSYGVHWIRQPPGKGLEWIGYIWAGGSINYNPSLKSRVTISVDTSKNQFSLKLSSVTAADTAVYYCARAYGDYVHYAMDYWGQGTLVTVSS

SEQ ID NO: 49 - protein, 120 aa, synthetic construct
QVQLQESGPGLVKPSETLSLTCTVSGFSLTSYGVHWIRQPPGKGLEWIGVIWAGGSINYNSALMSRVTISVDTSKNQFSLKLSSVTAADTAVYYCARAYGDYVHYAMDYWGQGTLVTVSS

SEQ ID NO: 50 - protein, 107 aa, synthetic construct
DIQMTQSPSSLSASVGDRVTITCRASQEISGYLTWYQQKPGKAPKLLIYAASTLDSGVPSRFSGSGSGTDFTLTISSLQPEDFATYYCLQYTNYPLTFGQGTKLEIK

SEQ ID NO: 51 - protein, 107 aa, synthetic construct
DIQMTQSPSSLSASVGDRVTITCRASQEISGYLTWLQQKPGKAPKLLIYAASTLDSGVPSRFSGSGSGTDFTLTISSLQPEDFATYYCLQYTNYPLTFGQGTKLEIK

SEQ ID NO: 52 - protein, 107 aa, synthetic construct
DIQMTQSPSSLSASVGDRVTITCRASQEISGYLTWFQQKPGKAPKSLIYAASTLDSGVPSRFSGSGSGTDFTLTISSLQPEDFATYYCLQYTNYPLTFGQGTKLEIK

SEQ ID NO: 53 - DNA, 1413 bp, synthetic construct
  source 1..1413: mol_type = other DNA
atggctctgctggtgctgtttctgagcctggccgcctttccttcttgtggcgtgctgtctcaggtgcagctgaaagagtctggccctggactggtggccccaagccagtctctgagcatcacctgtaccgtgtccggcttcagcctgacaagctatggcgtgcactgggtccgacagcctccaggcaaaggactggaatggctgggagtgatttgggctggcggcagcatcaactacaacagcgccctgatgagccggctgagcatctccaaggacaacttcaagagccaggtgttcctgaagatgagcagcctgcagaccgacgacaccgccatgtactattgcgccagagcctacggcgactacgtgcactacgccatggattattggggccagggcacaagcgtgacagccagctctgcctctacaaagggccctagcgttttcccactggctcccagcagcaagtctacatctggcggaacagccgctctgggctgcctggtcaaggattactttcccgagccagtgaccgtgtcctggaattctggcgctctgacaagcggcgtgcacacctttccagctgtgctgcaaagcagcggcctgtactctctgtccagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgctccgtgatgcacgaggccctgcacaaccactacacccagaagtccctgagcctgtctcctggcaagtga

SEQ ID NO: 54 - DNA, 711 bp, synthetic construct
  source 1..711: mol_type = other DNA
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaaccggggcgagtgttga

SEQ ID NO: 55 - DNA, 2112 bp, synthetic construct
  source 1..2112: mol_type = other DNA
atggctctgctggtgctgtttctgagcctggccgcctttccttcttgtggcgtgctgtctcaggtgcagctgaaagagtctggccctggactggtggccccaagccagtctctgagcatcacctgtaccgtgtccggcttcagcctgacaagctatggcgtgcactgggtccgacagcctccaggcaaaggactggaatggctgggagtgatttgggctggcggcagcatcaactacaacagcgccctgatgagccggctgagcatctccaaggacaacttcaagagccaggtgttcctgaagatgagcagcctgcagaccgacgacaccgccatgtactattgcgccagagcctacggcgactacgtgcactacgccatggattattggggccagggcacaagcgtgacagccagctctgcctctacaaagggccctagcgttttcccactggctcccagcagcaagtctacatctggcggaacagccgctctgggctgcctggtcaaggattactttcccgagccagtgaccgtgtcctggaattctggcgctctgacaagcggcgtgcacacctttccagctgtgctgcaaagcagcggcctgtactctctgtccagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgctccgtgatgcacgaggccctgcacaaccactacacccagaagtccctgagcctgtctcctggaaaaggcggcggaggaagtggtggcggaggatctagagttcagcccaccgagagcatcgtgcggttccccaacatcaccaatctgtgccctttcggcgaggtgttcaacgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcaccaacgtgtacgccgacagcttcgtgatcagaggcgacgaagtgcggcagattgcccctggacagacaggcaagatcgccgactacaattacaagctgcccgacgacttcaccggctgcgtgatcgcttggaacagcaacaacctggacagcaaagtcggcgggaactacaactacctgtatcggctgtttcggaagtccaacctgaagcctttcgagcgggacatcagcaccgagatctatcaggccggcagcaccccttgcaatggcgtcgaaggcttcaactgctacttcccactgcagtcctacggcttccagcctaccaatggcgtgggctaccagccttatagagtggtggtgctgagcttcgagctgctgcatgctcctgccacagtgtgcggccctaagaaaagcaccaatctcgtgaagaacaaatgcgtgaacttctga

SEQ ID NO: 56 - protein, 703 aa, synthetic construct
MALLVLFLSLAAFPSCGVLSQVQLKESGPGLVAPSQSLSITCTVSGFSLTSYGVHWVRQPPGKGLEWLGVIWAGGSINYNSALMSRLSISKDNFKSQVFLKMSSLQTDDTAMYYCARAYGDYVHYAMDYWGQGTSVTASSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 57 - DNA, 825 bp, synthetic construct
  source 1..825: mol_type = other DNA
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaacagaggcgagtgtggtggcagcggcggctctatcctgatgcagtacatcaaggccaacagcaagttcatcggcatccccatgggcctgcctcagtctattgctctgagcagcctgatggtggcccagtga

SEQ ID NO: 58 - protein, 274 aa, synthetic construct
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSILMQYIKANSKFIGIPMGLPQSIALSSLMVAQ

SEQ ID NO: 59 - DNA, 768 bp, synthetic construct
  source 1..768: mol_type = other DNA
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaacagaggcgagtgtggcggcagcggaggctctgctaaatttgtggctgcctggacactgaaggccgctgcttaa

SEQ ID NO: 60 - protein, 255 aa, synthetic construct
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSAKFVAAWTLKAAA

SEQ ID NO: 61 - DNA, 1410 bp, synthetic construct
  source 1..1410: mol_type = other DNA
atggacatgagagtgcctgctcacgtgttcggctttctgctgctgtggttccctggcaccagatgcgacatccagatgacacagagccccagcagcctgtctgcctctctgggacagagagtgtccctgacctgtagagccagccaagagatcagcggctacctgacatggctgcagcagaagcctgacggcaccatcaagagactggtgtacgccgccagcacactggatagcggagtgcccaagagattctccggcagcagatccggcagcgactacagcctgacaatcagctccctggaaagcgaggacttcgccgactactactgcctgcagtacacaaactaccctctgaccttcggagccggcaccaagctggaactgaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagtctgtgaccgagcaggacagcaaggactccacctatagcctgagcagcaccctgacactgagcaaggccgactacgagaagcacaaagtgtacgcctgcgaagtgacccaccagggcctttctagccctgtgaccaagagcttcaacagaggcgaatgtggcggcggaggatctggcggaggcggatctagagttcagcctaccgagagcatcgtgcggttccccaacatcaccaatctgtgccctttcggcgaggtgttcaacgccaccagattcgccagtgtgtatgcctggaaccggaagcggatcagcaactgcgtggccgattacagcgtgctgtacaacagcgccagcttcagcaccttcaagtgctacggcgtgtcccctaccaagctgaacgacctgtgcttcaccaacgtgtacgctgacagcttcgtgatccggggagatgaagtgcggcagattgcccctggacagacaggcaagattgccgactacaactacaagctgcccgacgacttcaccggctgtgtgatcgcctggaatagcaacaacctggacagcaaagtcggcggcaactacaattacctgtaccggctgttccggaagtccaacctgaagcctttcgagcgggacatcagcaccgagatctatcaggccggcagcaccccttgcaatggcgtggaaggcttcaactgctacttcccactgcagtcctacggcttccagcctacaaacggcgtgggctaccagccttacagagtggtggtgctgagcttcgagctgctgcatgctcctgccacagtgtgcggccctaagaaaagcaccaacctggtcaagaacaaatgcgtgaacttctga

SEQ ID NO: 62 - protein, 469 aa, synthetic construct
MDMRVPAHVFGFLLLWFPGTRCDIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 63 - protein, 107 aa, synthetic construct
DIQMTQSPSSLSASLGQRVSLTCRASQEISGYLTWLQQKPDGTIKRLVYAASTLDSGVPKRFSGSRSGSDYSLTISSLESEDFADYYCLQYTNYPLTFGAGTKLELK

SEQ ID NO: 64 - protein, 107 aa, synthetic construct; identical to SEQ ID NO: 45

SEQ ID NO: 65 - protein, 107 aa, synthetic construct; identical to SEQ ID NO: 46

SEQ ID NO: 66 - protein, 107 aa, synthetic construct; identical to SEQ ID NO: 47

SEQ ID NO: 67 - protein, 107 aa, synthetic construct; identical to SEQ ID NO: 50

SEQ ID NO: 68 - protein, 107 aa, synthetic construct; identical to SEQ ID NO: 51

SEQ ID NO: 69 - protein, 107 aa, synthetic construct; identical to SEQ ID NO: 52

(19) United States
(12) Patent Application Publication
(10) Pub. No.: US 2025/0312438 A1
(43) Pub. Date: Oct. 9, 2025

(54) HUMANIZED CONSTRUCTS, VACCINES, AND METHODS

(71) Applicant: THE HOSPITAL FOR SICK CHILDREN, Toronto (CA)

(72) Inventors: Jean-Philippe Julien, Toronto (CA); Arif Jetha, Toronto (CA); Audrey Kassardjian, Toronto (CA); Brian Barber, Toronto (CA)

(21) Appl. No.: 18/863,143

(22) PCT Filed: May 8, 2023
(86) PCT No.: PCT/CA2023/050622

§ 371 Date: Nov. 5, 2024

Related U.S. Application Data
(60) Provisional application No. 63/339,379, filed on May 6, 2022.

(51) Int. Cl. A61K 39/215; A61K 39/00; A61K 39/385; A61P 31/14; C07K 16/28; C12N 7/00

(52) U.S. Cl. CPC A61K 39/215; A61K 39/0016; A61K 39/385; A61P 31/14; C07K 16/2833; C12N 7/00; A61K 2039/575; A61K 2039/6056; C07K 2317/24; C07K 2317/30; C07K 2317/56

(57) ABSTRACT

Described herein in aspects is a humanized anti-class II MHC antibody, wherein the humanized antibody binds to class II MHC with similar or increased affinity and/or specificity as compared with a non-humanized anti-MHC class II antibody that specifically binds to a shared epitope on most or all HLA-DR molecules. Related methods and uses are also described.

### FIELD

The present invention relates to antibodies. In particular, the present invention relates to humanized antibodies, vaccines comprising the antibodies, and related compositions and methods.

### BACKGROUND

Humanized antibodies are antibodies from non-human species whose protein sequences have been modified to increase their similarity to antibody variants produced naturally in humans. The process of “humanization” is usually applied to monoclonal antibodies developed for administration to humans. Humanization can be necessary when the process of developing a specific antibody involves generation in a non-human immune system (such as that in mice). The protein sequences of antibodies produced in this way are partially distinct from homologous antibodies occurring naturally in humans, and are therefore potentially immunogenic when administered to human patients.

The ongoing Coronavirus Disease 2019 (COVID-19) pandemic, caused by the coronavirus designated SARS-CoV-2, has already (by April 2022) infected more than 500 million people, resulting in greater than 6 million deaths worldwide. A safe and effective vaccine which either prevents COVID-19, or substantially blunts its severity, is urgently required.

### SUMMARY OF THE INVENTION

In accordance with an aspect, there is provided a humanized anti-class II MHC antibody, wherein the humanized antibody binds to class II MHC with similar or increased affinity and/or specificity as compared with a non-humanized anti-MHC class II antibody that specifically binds to a shared epitope on most or all HLA-DR molecules.

In an aspect, the non-humanized anti-MHC class II antibody is based on 44H10.

In an aspect, the non-humanized MHC class II antibody is a human-mouse chimeric anti-class II MHC antibody based on 44H10.

In an aspect, the non-humanized MHC class II antibody comprises an amino acid sequence having at least 70% identity to SEQ ID NO. 31, 32, or a fragment thereof.

In an aspect, the antibody is an anti-HLA-DR antibody.

In an aspect, the antibody is broadly reactive with most or all HLA-DR molecules.

In an aspect, the antibody is an IgG, scFv, Fab′, Fab, F(ab′)₂, or scFab.

In an aspect, the antibody is an IgG.

In an aspect, the antibody is a monoclonal antibody.

In an aspect, the antibody has a 44H10 specificity.

In an aspect, the antibody comprises a VH construct comprising an amino acid sequence having at least 70% identity to SEQ ID NO. 33 or a fragment thereof.

In an aspect, the VH construct comprises a mutation that increases binding to HLA-DR.

In an aspect, the mutation is at position 71 and/or 78.

In an aspect, the mutation results in a K at position 71 and/or a V at position 78.

In an aspect, the antibody comprises a VL construct comprising an amino acid sequence having at least 70% identity to SEQ ID NO. 34, 35, 36, or a fragment thereof.

In an aspect, the VL construct comprises a mutation that increase binding to HLA-DR.

In an aspect, the mutation is at position 60 and/or 66.

In an aspect, the mutation results in a K at position 60 and/or an R at position 66.

In an aspect, the antibody is conjugated to another molecule.

In an aspect, the molecule comprises singularly or in combination a polypeptide or protein, a carbohydrate, a polynucleotide, a small molecule, or a lipid.

In an aspect, the polypeptide comprises an antigen.

In an aspect, the antigen is from an infectious agent.

In an aspect, the infectious agent is a coronavirus.

In an aspect, the coronavirus is SARS-CoV-1, SARS-CoV-2, or MERS.

In an aspect, the coronavirus antigen is a spike protein antigen or a nucleocapsid antigen.

In an aspect, the coronavirus antigen is an S1 antigen or an S2 antigen.

In an aspect, the coronavirus antigen is an RBD antigen.

In an aspect, the coronavirus antigen comprises a polypeptide having at least 70% identity to SEQ ID NO. 37, 38, 39, 40, 41, or a fragment thereof.

In an aspect, the molecule is conjugated to a heavy chain of the antibody.

In an aspect, the molecule is conjugated at the C-terminus of the heavy chain.

In an aspect, the molecule is conjugated to a light chain of the antibody.

In an aspect, the molecule is conjugated at the C-terminus of the light chain.

In an aspect, the antibody comprises two heavy chains and/or two light chains and a plurality of molecules, either the same or different, each conjugated to a different heavy chain and/or light chain.

In an aspect, the antibody comprises two or more molecules, each conjugated to a respective heavy chain or light chain, wherein the molecules are independently the same or different.

In an aspect, the antibody comprises three or four molecules, each conjugated to a respective heavy chain or light chain, wherein the molecules are independently the same or different.

In an aspect, the antibody comprises four molecules, one at each heavy and light chain.

In an aspect, the molecules at each heavy chain are the same and the molecules at each light chain are the same, and wherein the molecules at each heavy chain are different from the molecules at each light chain.

In an aspect, the antibody comprises two molecules, each conjugated to a respective heavy chain.

In an aspect, the antibody further comprises a linker between the antibody and the molecule.

In an aspect, the linker is a GS repeat linker, such as a GGSx2 linker or GGGGSx2 linker.

In an aspect, the molecule is a universal T-helper determinant.

In an aspect, the universal T-helper determinant comprises PADRE and/or TpD.

In an aspect, the universal T-helper determinant is conjugated to a heavy chain of the antibody.

In an aspect, the universal T-helper determinant is conjugated at the C-terminus of the heavy chain.

In an aspect, the universal T-helper determinant is conjugated to a light chain of the antibody.

In an aspect, the universal T-helper determinant is conjugated at the C-terminus of the light chain.

In an aspect, the antibody comprises two heavy chains and/or two light chains and comprising a plurality of universal T-helper determinants, either the same or different, each conjugated to a different heavy chain and/or light chain.

In an aspect, the antibody comprises two universal T-helper determinants, each conjugated to a respective heavy chain or light chain.

In an aspect, the antibody comprises two universal T-helper determinants, each conjugated to a respective light chain.

In an aspect, the antibody further comprises a linker between the antibody and the universal T-helper determinant.

In an aspect, the linker is a GS repeat linker, such as a GGSx2 linker or GGGGSx2 linker.

In an aspect, the antibody is for use in combination with a vaccine against tetanus and/or diphtheria toxoids.

In an aspect, the antibody comprises a polypeptide sequence having at least 70% sequence identity to any one or more of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In an aspect, the antibody comprises at least one heavy chain and at least one light chain of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, in any combination.

In an aspect, the antibody comprises two heavy chains and two light chains of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, in any combination.

In an aspect, the antibody consists of two heavy chains and two light chains of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, in any combination.

In accordance with an aspect, there is provided a polynucleotide encoding the antibody described herein.

In accordance with an aspect, there is provided a vaccine comprising the polynucleotide described herein.

In accordance with an aspect, there is provided a vector comprising the polynucleotide described herein.

In accordance with an aspect, there is provided a host cell comprising the vector described herein.

In accordance with an aspect, there is provided a vaccine comprising the antibody described herein.

In an aspect, the vaccine is free of an adjuvant.

In accordance with an aspect, there is provided a method of immunizing a subject against a disease or condition, the method comprising administering the vaccine described herein to the subject.

In accordance with an aspect, there is provided a method of treating and/or preventing a disease or condition in a subject, the method comprising administering the vaccine described herein to the subject.

In an aspect, the method further comprising administering a vaccine against tetanus and/or diphtheria toxoids to the subject.

In an aspect, the vaccine against tetanus and/or diphtheria toxoids is administered prior to administering the vaccine against the disease or condition.

In an aspect, the vaccine against tetanus and/or diphtheria toxoids is administered to the subject prior to the vaccine against the disease or condition, such as one or more days, weeks, months, or years prior to the vaccine against the disease or condition, such as about one month prior to the vaccine against the disease or condition.

In an aspect, the vaccine against the disease or condition is administered without an adjuvant.

In an aspect, the vaccine against the disease or condition is administered as a purified protein without an adjuvant.

In accordance with an aspect, there is provided a use of the vaccine described herein for immunizing a subject against a disease or condition.

In accordance with an aspect, there is provided a use of the vaccine described herein for treating and/or preventing a disease or condition.

In an aspect, the use further comprises use of a vaccine against tetanus and/or diphtheria toxoids.

In an aspect, the vaccine against tetanus and/or diphtheria toxoids is for use prior to the vaccine against the disease or condition, such as one more days, weeks, months, or years prior to the vaccine against the disease or condition, such as about one month prior to the vaccine against the disease or condition.

In an aspect, the vaccine against the disease or condition is for use without an adjuvant.

In an aspect, the vaccine against the disease or condition is for use as a purified protein without an adjuvant.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figures, in which:

FIG. 1 shows a schematic representation of the chimeric human IgG1 44H10 anti-HLA-DR antibody and four different mAbs (0, 1, 2 and 3) based on the immunotargeting vector described herein.

FIG. 2A shows the DNA sequence and derived amino acid sequence of the variable region of the murine 44H10 mAb heavy chain (VH) and human IgG1 constant region, used in the expression of the chimeric human IgG1 antibody with 44H10 specificity (Chi-44H10). Each feature of the sequence is identified by annotated boxes.

FIG. 2B shows the DNA sequence and derived amino acid sequence of the variable region of the murine 44H10 mAb light chain (VL) and human kappa constant region, used in the expression of Chi-44H10 and mAb 0. Each feature of the sequence is identified by annotated boxes.

FIG. 2C shows the DNA sequence and derived amino acid sequence of the chimeric mouse/human IgG1 heavy chain used in the expression of mAbs 0, 1, 2, and 3, with the SARS-CoV-2 Spike protein RBD linked to the C-terminus of the heavy chain by a short linker peptide. Each feature of the sequence is identified by annotated boxes.

FIG. 2D shows the DNA sequence and derived amino acid sequence of the chimeric mouse/human kappa (κ) light chain used in the expression of mAb 1, with the T helper determinant TpD linked to the C-terminus of the light chain by a short linker peptide. Each feature of the sequence is identified by annotated boxes.

FIG. 2E shows the DNA sequence and derived amino acid sequence of the chimeric mouse/human kappa (κ) light chain used in the expression of mAb 2, with the T helper determinant PADRE linked to the C-terminus of the light chain by a short linker peptide. Each feature of the sequence is identified by annotated boxes.

FIG. 2F shows the DNA sequence and derived amino acid sequence of the chimeric mouse/human kappa (κ) light chain used in the expression of mAb 3, with the SARS-CoV-2 Spike protein RBD linked to the C-terminus of the light chain by a short linker peptide. Each feature of the sequence is identified by annotated boxes.

FIG. 3 shows representative plasmid maps of the pcDNA3.4 TOPO vector used to express the chimeric 44H10 heavy and light chains in the FreeStyle 293-F cell line. Segments of the plasmids shown in red correspond to the DNA encoding each chain of the chimeric 44H10 antibody. Heavy and light chains of the immunotargeting mAbs were expressed in the same expression vector.

FIG. 4A shows the elution profiles of Chi-44H10 and immunotargeting mAbs purified by protein A chromatography.

FIG. 4B depicts Coomassie Blue-stained SDS-PAGE 4-20% gradient gels. Each purified mAb was run on the gels under non-reducing (NR) and reducing (R) conditions. Bands in the non-reducing condition correspond to intact Chi-44H10 or immunotargeting mAbs, and bands in the reducing condition correspond to the heavy and light chains of each mAb.

FIG. 5 shows flow cytometry data demonstrating the binding of chimeric 44H10 antibody conjugates to the lymphoblastoid B cell line BJAB. Chi-44H40 and immunotargeting mAbs were directly labeled using an Alexa 488 (A488) Maleimide dye and the binding was measured in the B530 channel. Gates on the histograms represent the positive signal established cells treated with the positive control (anti-CD19 antibody Denintuzumab).

FIG. 6A shows a structural model depicting the binding of three antibodies (CR3022, S309 and VHH-72) to three distinct conformational epitopes on the SARS-CoV-2 spike protein RBD. The Protein Data Bank (PDB) identification number corresponding to each antibody-RBD complex is specified next to each antibody.

FIG. 6B shows flow cytometry data demonstrating the binding of the three aforementioned antibodies to the RBD displayed on the immunotargeting mAbs. The immunotargeting mAbs were allowed to bind to BJAB cells, and then reacted with the anti-RBD antibodies fluorescently labeled using an Alexa 488 Maleimide dye. The binding was measured in the B530 channel. Gates on the histograms represent the positive signal established cells treated with the positive control (anti-CD19 antibody Denintuzumab).

FIG. 7A shows ELISA endpoint titer analysis depicting between immunization group comparisons of antibody titers at D49 elicited by soluble RBD (sRBD), Chi-44H10 and immunotargeting mAbs.

FIG. 7B shows ELISA data depicting the kinetics of anti-RBD antibody responses elicited by sRBD, Chi-44H10 and immunotargeting mAbs.

FIG. 8A shows SARS-CoV-2 Spike protein-expressing pseudovirus (wild-type) neutralization data comparing neutralization potency of serum antibodies elicited in rabbits immunized with sRBD, Chi-44H10 or immunotargeting mAbs at D49, D70 and D91. These data were fitted by non-linear regression.

FIG. 8B shows SARS-CoV-2 Spike protein-expressing pseudovirus neutralization data comparing neutralization potency of D49 serum antibodies elicited in rabbits immunized with immunotargeting mAbs against WIV04/2019 (wild-type), B.1.351 (beta), P.1 (gamma) and B.1.617.2 (delta) strains of SARS-CoV-2. These data were fitted by non-linear regression.

FIG. 9A shows ELISA data depicting the kinetics of anti-RBD antibody responses elicited by immunotargeting mAbs administered either subcutaneously (sub-Q) or intramuscularly (IM).

FIG. 9B shows SARS-CoV-2 Spike protein-expressing pseudovirus (wild-type) neutralization data comparing neutralization potency of serum antibodies elicited in rabbits immunized with immunotargeting mAbs administered either subcutaneously (sub-Q) or intramuscularly (IM) at D49, D70 and D91. These data were fitted by non-linear regression.

FIG. 10 shows a flow cytometric measurement of first-round humanized 44H10 variants (V1-V9) binding to BJAB cells at 10 μg/ml, compared to the parental chimeric 44H10 antibody.

FIG. 11 shows a flow cytometric measurement of second-round humanized 44H10 variants (V10-V18) binding to BJAB cells at a concentration of 10 μg/ml, compared to the parental chimeric 44H10 antibody.

FIG. 12 shows the co-crystal structure of the 44H10 Fab in complex with HLA-DR (HLA-DRA*01:01, HLA-DRB1*04:01) solved to 3.1 Å resolution, revealing critical contributions from the key light chain framework residues K60 and R66 to the Fab: HLA-DR interaction.

FIG. 13 shows a sequence alignment comparing the VL of parental VL 44H10 to humanized constructs VL1-VL6. Dashed lines denote CDR definitions based on the KABAT scheme whereas solid lines represent CDR definitions based on the IMGT definition. K60S and R66G mutations are highlighted by red arrows.

FIG. 14 shows a flow cytometric measurement of humanized 44H10 variants designed with incorporation of structure-guided principles, V19 and V20, binding to BJAB cells, compared to the second-round V17 humanized variant evolved from simple CDR grafting approaches, with the accompanying table of IC50s and IC90s.

FIG. 15 shows (A) BLI measurement of parental and humanized V21 44H10 antibodies binding to recombinant HLA-DR, with accompanying table comparing binding kinetics and (B) flow cytometric measurement of V21 binding to BJAB cells at a concentration of 7.81 μg/ml, compared to the parental chimeric 44H10 antibody.

FIG. 16 shows (A) the co-crystal structure of the HLA-DR a chain, β chain, the HA peptide, 44H10 VH and 44H10 V_{K} and (B) BLI measurement of parental and humanized 44H10 antibodies binding to recombinant HLA-DR.

FIG. 17 shows binding of 44H10 to donor PBMC samples.

FIG. 18 shows results of immunization in rabbits, where (A) shows the immunization schedule, (B) shows the anti-RBD antibody titers over time, and (C) shows percent neutralization at different timepoints.

FIG. 19 shows day 49 percent neutralization after immunization in rabbits comparing the immunotargeting mAb, the immunotargeting mAb with TpD, and the neutralizing therapeutic antibody REGN10987.

FIG. 20 shows that (A) adjuvant-free immunization with the immunotargeting mAb (B) induced robust anti-RBD IgG titers in and (C) and (D) neutralized virus in vivo following challenge (E) resulting in improved clinical scores post-challenge.

FIG. 21 shows (A) vaccine design and (B), (C), and (D) show characterization of the mono-antigenic design and the bi-antigenic design, (E) shows the virus neutralizing ability of the modular immunotargeting vaccine against various sarbecoviruses.

FIG. 22 shows that VH residues at framework positions 71 and 78 impact HCDR1 conformation.

FIG. 23 shows that (A) and (B) mutating K71 to a V in V21 (binder) completely knocked out binding to recombinant HLA-DR, (E) and (F) mutating V71 to a K in V14 (non-binder) restored some HLA-DR binding, (A) and (C) steric hindrance caused by the V78F mutation in V21 on the K71-L29 interaction significantly reduced binding, (E) and (G) removal of this steric hindrance in V14 through the F78V mutation was unable to restore binding, (E) and (H) the V71K and F78V mutations restore V14 binding at an affinity almost equivalent to V21.

### DETAILED DESCRIPTION OF CERTAIN ASPECTS

Described herein is a humanized anti-class II MHC antibody. Antibody humanization is a method used to reduce the amount of foreign content in antibodies generated from immunized hosts, such as rodents and rabbits. The ultimate goal of humanization is to prevent unwanted immunogenicity against the molecule in humans, while retaining the affinity and specificity of the parental non-human antibody. The antibody can be linked to a molecule, such as an antigen, to create a vaccine construct that is highly thermostable. When injected, this vaccine construct induces a potent, long-lived, neutralizing IgG antibody response. In certain specific aspects, these vaccine constructs utilize a humanized recombinant monoclonal antibody (mAb) specific for a serological determinant widely expressed on human class II major histocompatibility complex (MHC) gene products. The molecule, such as an antigen, such as the receptor binding domain (RBD) of the SARS-CoV-2 virus Spike protein is genetically incorporated into the immunotargeting antibody to create the vaccine construct. In addition, specific sequences corresponding to universal T-helper determinants are also typically incorporated into the vaccine constructs. The results described herein indicate that rabbits and ferrets immunized with these specific constructs induce potent and long-lived antibody responses which (using in vitro cellular infection assays) can be shown to neutralize virus expressing the corresponding SARS-CoV-2 Spike protein. The vaccines described herein protected ferrets from SARS-CoV-2 infection and broadly neutralized against wild-type SARS-CoV-2 as well as variants of concern. The results herein also indicate that certain anti-viral antibody responses are unexpectedly dependent on the specific sites of incorporation of the viral RBD and the T-helper sequences.

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only, and is not intended to be limiting. Many patent applications, patents, and publications are referred to herein to assist in understanding the aspects described. Each of these references are incorporated herein by reference in their entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight, typically less than 3% by weight, more typically less than 1%, and even more typically less than 0.1% by weight of non-specified component(s).

It will be understood that any component defined herein as being included may be explicitly excluded from the claimed invention by way of proviso or negative limitation. For example, in aspects, the compositions and vaccines described herein are free of an adjuvant or “adjuvant-free.”

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.” The word “or” is intended to include “and” unless the context clearly indicates otherwise.

A “vaccine” is a pharmaceutical composition that induces a prophylactic or therapeutic immune response in a subject. In some cases, the immune response is a protective immune response. Typically, a vaccine induces an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In one specific, non-limiting example, a vaccine induces an immune response that reduces the severity of the symptoms associated with SARS-CoV-2 infection and/or decreases the viral load compared to a control. In another non-limiting example, a vaccine induces an immune response that reduces and/or prevents SARS-CoV-2 infection compared to a control.

The term “antibody”, also referred to in the art as “immunoglobulin” (Ig), used herein refers to a protein constructed from paired heavy and light polypeptide chains; various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable (V_{L}) and a constant (C_{L}) domain, while the heavy chain folds into a variable (V_{H}) and three constant (C_{H}, C_{H2}, C_{H3}) domains. Interaction of the heavy and light chain variable domains (V_{H} and V_{L}) results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important immunological events. The variable region of an antibody contains the antigen binding determinants of the molecule, and thus determines the specificity of an antibody for its target antigen. The majority of sequence variability occurs in six hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape and chemistry of the surface they present to the antigen.

An “antibody fragment” as referred to herein may include any suitable antigen-binding antibody fragment known in the art. The antibody fragment may be a naturally-occurring antibody fragment, or may be obtained by manipulation of a naturally-occurring antibody or by using recombinant methods. For example, an antibody fragment may include, but is not limited to a Fv, single-chain Fv (scFv; a molecule consisting of V_{L} and V_{H} connected with a peptide linker), Fab, F(ab′)₂, single domain antibody (sdAb; a fragment composed of a single V_{L} or V_{H}), and multivalent presentations of any of these.

By the term “synthetic antibody” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “humanized antibody” as used herein includes antibodies in which CDR sequences derived from the germline of another mammalian species, such as a mouse, have been grafted onto human framework sequences based on sequence or structural similarity. Additional framework region modifications may be made within the human framework sequences as well as within the CDR sequences derived from the germline of another mammalian species.

The term “epitope” refers to an antigenic determinant. An epitope is the particular chemical groups or peptide sequences on a molecule that are antigenic, that is, that elicit a specific immune response. An antibody specifically binds a particular antigenic epitope, e.g., on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, about 11, or about 8 to about 12 amino acids in a unique spatial conformation. Methods of determining spatial conformation of epitopes include, for example, x-ray crystallography and 2-dimensional nuclear magnetic resonance. See, e.g., “Epitope Mapping Protocols” in Methods in Molecular Biology, Vol. 66, Glenn E. Morris, Ed (1996).

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the aspects described herein include, but are not limited to, the use of partial nucleotide sequences of more than one gene and that these nucleotide sequences could be arranged in various combinations to elicit the desired immune response. Moreover, a skilled artisan will understand that an antigen need not be encoded by a “gene” at all. It is readily apparent that an antigen can be synthesized or can be derived from a biological sample. Such a biological sample can include, but is not limited to a tissue sample, a cell, or a biological fluid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “purified” means that impurities have been removed and the purified component is present at a higher concentration than it would otherwise be. For example, a composition comprising a purified component may comprise 85%, 90%, 95%, 96%, 97%, 98%, 99%, 99.5%, 99.9% or more of the component or 100% of the component in question.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, typically, a human.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

Furthermore, the term “broadly reactive” means that the antibody reacts or binds to a common (shared) genetic determinant or epitope expressed on multiple HLA-DR alleles in the human population. For example, the antibody may bind to any one or more of HLA-DR1, HLA-DR3, HLA-DR4, HLA-DR7, HLA-DR8, HLA-DR9, HLA-DR10, HLA-DR11, HLA-DR12, HLA-DR13, HLA-DR14, HLA-DR15, and/or HLA-DR 16.

The terms “therapeutically effective amount”, “effective amount” or “sufficient amount” mean a quantity sufficient, when administered to a subject, including a mammal, for example a human, to achieve a desired result, for example an amount effective to cause a protective immune response. Effective amounts of the compounds described herein may vary according to factors such as the immunogen, age, sex, and weight of the subject. Dosage or treatment regimes may be adjusted to provide the optimum therapeutic response, as is understood by a skilled person. For example, administration of a therapeutically effective amount of the antibodies described herein is, in aspects, sufficient to increase immunity against a pathogen, such as SARS-CoV-2.

Moreover, a treatment regime of a subject with a therapeutically effective amount may consist of a single administration, or alternatively comprise a series of applications. The length of the treatment period depends on a variety of factors, such as the immunogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The antibodies described herein may, in aspects, be administered before, during or after treatment with conventional therapies for the disease or disorder in question.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable carrier” includes, but is not limited to solvents, dispersion media, coatings, antibacterial agents, antifungal agents, isotonic and/or absorption delaying agents and the like. The use of pharmaceutically acceptable carriers is well known.

The term “adjuvant” refers to a compound or mixture that is present in a vaccine and enhances the immune response to an antigen present in the vaccine. For example, an adjuvant may enhance the immune response to a polypeptide present in a vaccine as contemplated herein, or to an immunogenic fragment or variant thereof as contemplated herein. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL 1005), aluminum phosphates (e.g., AIPO.sub.4), R-848 (a Th1-like adjuvant), imiquimod, PAM3CYS, poly (I: C), loxoribine, BCG (bacille Calmette-Guerin) and Corynebacterium parvum, CpG oligodeoxynucleotides (ODN), cholera toxin derived antigens (e.g., CTA 1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

“Variants” are biologically active proteins, antibodies, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

“Active” or “activity” for the purposes herein refers to a biological and/or an immunological activity of the antibodies described herein, wherein “biological” activity refers to a biological function (either inhibitory or stimulatory) caused by the antibodies.

The proteins described herein may include modifications. Such modifications include, but are not limited to, conjugation to an effector molecule such as an anti-viral agent or an adjuvant. Modifications further include, but are not limited to conjugation to detectable reporter moieties. Modifications that extend half-life (e.g., pegylation) are also included. Proteins and non-protein agents may be conjugated to the antibodies by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

### Antibodies

In aspects, described herein is a humanized anti-class II MHC antibody. The humanized antibody binds to class-II MHC with similar or increased affinity and/or specificity as compared with a non-humanized anti-MHC class II antibody that specifically binds to a shared epitope on most or all HLA-DR molecules.

In aspects, the non-humanized anti-MHC class II antibody is based on 44H10, for example it may be a fully mouse antibody or some other species or is may be a chimeric antibody, such as a human-mouse chimeric anti-class II MHC antibody based on 44H10.

In aspects, the non-humanized MHC class II antibody comprises an amino acid sequence having at least 70% identity to:

SEQ ID NO. 31: Chimeric 44H10 heavy chain
MALLVLFLSLAAFPSCGVLSQVQLKESGPGLVAPSQSLSITCTVSG
FSLTSYGVHWVRQPPGKGLEWLGVIWAGGSINYNSALMSRLSISKD
NFKSQVFLKMSSLQTDDTAMYYCARAYGDYVHYAMDYWGQGTSVTA
SSASTKGPSVFPLAPSSKSTSGGTAALGCLVKDYFPEPVTVSWNSG
ALTSGVHTFPAVLQSSGLYSLSSVVTVPSSSLGTQTYICNVNHKPS
NTKVDKKVEPKSCDKTHTCPPCPAPELLGGPSVFLFPPKPKDTLMI
SRTPEVTCVVVDVSHEDPEVKFNWYVDGVEVHNAKTKPREEQYNST
YRVVSVLTVLHQDWLNGKEYKCKVSNKALPAPIEKTISKAKGQPRE
PQVYTLPPSRDELTKNQVSLTCLVKGFYPSDIAVEWESNGQPENNY
KTTPPVLDSDGSFFLYSKLTVDKSRWQQGNVFSCSVMHEALHNHYT
QKSLSLSPGK▮
Signal peptide
44H10 V_{H}
Human C_{H} (IgG₁)
▮
SEQ ID NO. 32: Chimeric 44H10 light chain
MDMRVPAHVFGFLLLWFPGTRCDIQMTQSPSSLSASLGQRVSLTCR
ASQEISGYLTWLQQKPDGTIKRLVYAASTLDSGVPKRFSGSRSGSD
YSLTISSLESEDFADYYCLQYTNYPLTFGAGTKLELKRTVAAPSVF
IFPPSDEQLKSGTASVVCLLNNFYPREAKVQWKVDNALQSGNSQES
VTEQDSKDSTYSLSSTLTLSKADYEKHKVYACEVTHQGLSSPVTKS
FNRGEC▮
Signal peptide
44H10 V_{K}
Human C_{K}
▮

or a fragment thereof.

Typically, the antibody is an anti-HLA-DR antibody. In typical aspects, the antibody is a broadly reactive anti-HLA-DR antibody. The antibody may be of any form or fragment but is typically an IgG, scFv, Fab′, Fab, F(ab′)₂, or scFab antibody. Typically, the antibody is an IgG antibody.

In typical aspects, the antibody is a humanized monoclonal antibody, such as a 44H10 antibody, which specifically binds to a shared epitope on most or all HLA-DR molecules. Any other monoclonal antibody that has this same specificity could substitute for the 44H10 antibody.

In typical aspects, the antibody comprises a V_{H} construct comprising an amino acid sequence having at least 70% identity to SEQ ID NO. 33 or a fragment thereof.

In additional or alternative aspects, the antibody typically comprises a V_{L} construct comprising an amino acid sequence having at least 70% identity to SEQ ID NO. 34, 35, 36 or a fragment thereof.

It will be understood that the antibody described herein may be used alone or in conjunction with other molecules. For example, the antibody may be conjugated to another molecule. The molecule is without limitation by may comprise, for example, singularly or in combination a polypeptide or protein, a carbohydrate, a polynucleotide, a small molecule, or a lipid. In typical aspects, the polypeptide comprises an antigen. The antigen may be from any condition or disease known to be improved, treated, or prevented by vaccination. In typical aspects, the antigen is from an infectious agent. While all infectious agents are contemplated, typically the infectious agent is a coronavirus, such as SARS-CoV-1, SARS-CoV-2, or MERS. The coronavirus antigen may be any antigen from the coronavirus, such as a spike protein antigen or a nucleocapsid antigen. For example, the coronavirus antigen may be a spike protein S1 antigen or an S2 antigen. Typically, the coronavirus antigen is an RBD antigen. It will be understood that the coronavirus antigens described herein may be derived from any variant of any coronavirus. In specific reference to SARS-CoV-2, the SARS-CoV-2 antigens described herein may be derived from any variant of SARS-CoV-2, including alpha, beta, delta, omicron, etc., as well as any subvariants thereof, such as omicron BA.1, BA.2, and so on. In aspects, the antigens may be conserved between variants or may be variant-specific.

In certain typical aspects, the coronavirus antigen comprises a polypeptide having at least 70% identity to:

SEQ ID NO. 37: WuhanRBD
RVQPTESIVRFPNITNLCPFGEVFNATRFASVYAWNRKRISNCVAD
YSVLYNSASFSTFKCYGVSPTKLNDLCFTNVYADSFVIRGDEVRQI
APGQTGKIADYNYKLPDDFTGCVIAWNSNNLDSKVGGNYNYLYRLF
RKSNLKPFERDISTEIYQAGSTPCNGVEGFNCYFPLQSYGFQPTNG
VGYQPYRVVVLSFELLHAPATVCGPKKSTNLVKNKCVNF
SEQ ID NO. 38: WuhanRBD V2 (C-term trunc)
RVQPTESIVRFPNITNLCPFGEVFNATRFASVYAWNRKRISNCVAD
YSVLYNSASFSTFKCYGVSPTKLNDLCFTNVYADSFVIRGDEVRQI
APGQTGKIADYNYKLPDDFTGCVIAWNSNNLDSKVGGNYNYLYRLF
RKSNLKPFERDISTEIYQAGSTPCNGVEGFNCYFPLQSYGFQPTNG
VGYQPYRVVVLSFELLHAPATVCGPKKS
SEQ ID NO. 39: WuhanRBD V3 (N- and C-term trunc)
TNLCPFGEVFNATRFASVYAWNRKRISNCVADYSVLYNSASFSTFK
CYGVSPTKLNDLCFTNVYADSFVIRGDEVRQIAPGQTGKIADYNYK
LPDDFTGCVIAWNSNNLDSKVGGNYNYLYRLFRKSNLKPFERDIST
EIYQAGSTPCNGVEGFNCYFPLQSYGFQPTNGVGYQPYRVVVLSFE
LLHAPATVCGPKKS
SEQ ID NO. 40: OmicronRBD V2 (C-term trunc)
RVQPTESIVRFPNITNLCPFDEVFNATRFASVYAWNRKRISNCVAD
YSVLYNLAPFFTFKCYGVSPTKLNDLCFTNVYADSFVIRGDEVRQI
APGQTGNIADYNYKLPDDFTGCVIAWNSNKLDSKVSGNYNYLYRLF
RKSNLKPFERDISTEIYQAGNKPCNGVAGFNCYFPLRSYSFRPTYG
VGHQPYRVVVLSFELLHAPATVCGPKKS
SEQ ID NO. 41: CoV1RBD
TNLCPFGEVFNATKFPSVYAWERKKISNCVADYSVLYNSTFFSTFK
CYGVSATKLNDLCFSNVYADSFVVKGDDVRQIAPGQTGVIADYNYK
LPDDFMGCVLAWNTRNIDATSTGNYNYKYRYLRHGKLRPFERDISN
VPFSPDGKPCTPPALNCYWPLNDYGFYTTTGIGYQPYRVVVLSFEL
LNAPATVCGPK

or a fragment thereof.

It will be understood that the molecule may be conjugated to any part of the antibody, although typically it is conjugated away from the N-terminus to avoid inhibiting the antigen binding ability of the antibody. In typical aspects, the molecule is conjugated at or near the C-terminus of the heavy and/or light chain of the antibody. In some aspects, the molecule is conjugated to the heavy chain of the antibody and in some aspects, the molecule is conjugated to the light chain of the antibody. In typical aspects, wherein the antibody comprises two heavy chains and two light chains, a molecule is conjugated to each heavy chain or each light chain. In this case, the molecule may be independently the same or different but is typically the same. In other aspects, a molecule may be conjugated to one heavy chain and one light chain, two heavy chains, two light chains, all four heavy and light chains, or various combinations thereof. In other aspects, a plurality of molecules, being the same or different, may be conjugated to one or more antibody heavy or light chains, in series or parallel. Typically, the molecule is a coronavirus antigen and typically the coronavirus antigen is the RBD and typically an RBD is conjugated to each heavy chain at the C-terminus thereof. The RBD antigens may be the same or different and there may be 1, 2, 3, or 4 same or different antigens, such as RBD antigens. In some aspects, antigens from different variants are combined in a single antibody so as to target multiple variants simultaneously and to be broadly neutralizing. In some aspects, antigens from different targets are combined.

Other moieties may be additionally conjugated to the antibody described herein. For example, the antibody may be conjugated to a universal T-helper determinant. Examples of a universal T-helper determinant include PADRE and/or TpD. Similar to the coronavirus antigen, the universal T-helper determinant is typically conjugated at or near the C-terminus of the heavy chain and/or light chain of the antibody. In some aspects, the universal T-helper determinant is conjugated to the heavy chain of the antibody and in some aspects, the universal T-helper determinant is conjugated to the light chain of the antibody. In typical aspects, wherein the antibody comprises two heavy chains and two light chains, a universal T-helper determinant is conjugated to each heavy chain or each light chain. In this case, the universal T-helper determinant may be the same or different but is typically the same. In other aspects, a universal T-helper determinant may be conjugated to one heavy chain and one light chain, two heavy chains, two light chains, all four heavy and light chains, or various combinations thereof. In other aspects, a plurality of universal T-helper determinant antigens, being the same or different, may be conjugated to one or more antibody heavy or light chains, in series or parallel. Typically, the universal T-helper determinant is the PADRE sequence and typically a PADRE sequence is conjugated to each light chain at the C-terminus thereof. The universal T-helper determinants may be the same or different and there may be 1, 2, 3, or 4 same or different universal T-helper determinants, such as PADRE sequences. It will be understood that the universal T-helper determinants may be bound directly to the antibody or to another molecule that is itself bound to the antibody, directly or via a linker. For example, an antigen may be bound to the light chain and the universal T-helper determinant may be bound to the antigen. This demonstrates how modular this system is, in that the various molecules bound to the antibody can be placed in different locations and/or orders as determined by the desired end use.

It will be understood that linkers may be included that separate the antibody from another bound moiety, such as between the antibody and the coronavirus antigen or the antibody and the universal T-helper determinant or between the antigen and the universal T-helper determinant. Typically, the linker is a GS repeat linker, such as a GGSx2 linker or GGGGSx2 linker.

In typical aspects, the antibody comprises a polypeptide sequence having at least 70% sequence identity to any one or more of the following:

Humanized HC-WuhanRBD
SEQ ID NO. 1
MEFGLSWVFLVALFRGVQSQVTLKESGPVLVKPTETLTLTCTVSGFSLTSYGVHWIRQPPGKALEWL
AVIWAGGSISYSTSLMSRLTISKDTSKSQVVLTMTNMDPVDTATYYCARAYGDYVHYAMDYWGQGT
LVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGGGGSGGGGSRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSYGFQPTNGVGYQP
YRVVVLSFELLHAPATVCGPKKSTNLVKNKCVNF▮
Signal peptide
Humanized 44H10 V_{H}
Human C_{H} (IgG₁)
Linker
RBD (319-542)
▮
Humanized HC-WuhanRBD V2 (C-term trunc)
SEQ ID NO. 2
MEFGLSWVFLVALFRGVQSQVTLKESGPVLVKPTETLTLTCTVSGFSLTSYGVHWIRQPPGKALEWL
AVIWAGGSISYSTSLMSRLTISKDTSKSQVVLTMTNMDPVDTATYYCARAYGDYVHYAMDYWGQGT
LVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGGGGSGGGGSRVQPTESIVRFPNITNLCPFGEVFNATRFASVYAWNRKRISNCVADYSVLYNSA
SFSTFKCYGVSPTKLNDLCFTNVYADSFVIRGDEVRQIAPGQTGKIADYNYKLPDDFTGCVIAWNSNN
LDSKVGGNYNYLYRLFRKSNLKPFERDISTEIYQAGSTPCNGVEGFNCYFPLQSYGFQPTNGVGYQP
YRVVVLSFELLHAPATVCGPKKS▮
Signal peptide
Humanized 44H10 V_{H}
Human C_{H} (IgG₁)
Linker
RBD (319-531)
▮
Humanized HC-WuhanRBD V3 (N- and C-term trunc)
SEQ ID NO. 3
MEFGLSWVFLVALFRGVQSQVTLKESGPVLVKPTETLTLTCTVSGFSLTSYGVHWIRQPPGKALEWL
AVIWAGGSISYSTSLMSRLTISKDTSKSQVVLTMTNMDPVDTATYYCARAYGDYVHYAMDYWGQGT
LVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGGGGSGGGGSTNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTNGVGYQPYRVVVLSFELLHA
PATVCGPKKS▮
Signal peptide
Humanized 44H10 V_{H}
Human C_{H} (IgG₁)
Linker
RBD (333-530)
▮
Humanized HC-TpD
SEQ ID NO. 4
MEFGLSWVFLVALFRGVQSQVTLKESGPVLVKPTETLTLTCTVSGFSLTSYGVHWIRQPPGKALEWL
AVIWAGGSISYSTSLMSRLTISKDTSKSQVVLTMTNMDPVDTATYYCARAYGDYVHYAMDYWGQGT
LVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGGSGGSILMQYIKANSKFIGIPMGLPQSIALSSLMVAQ▮
Signal peptide
Humanized 44H10 V_{H}
Human C_{H} (IgG₁)
Linker
TpD
▮
Humanized HC-PADRE
SEQ ID NO. 5
MEFGLSWVFLVALFRGVQSQVTLKESGPVLVKPTETLTLTCTVSGFSLTSYGVHWIRQPPGKALEWL
AVIWAGGSISYSTSLMSRLTISKDTSKSQVVLTMTNMDPVDTATYYCARAYGDYVHYAMDYWGQGT
LVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGGSGGSAKFVAAWTLKAAA▮
Signal peptide
Humanized 44H10 V_{H}
Human C_{H} (IgG₁)
Linker
PADRE
▮
Humanized HC-OmicronRBD-TpD V2 (C-term trunc)
SEQ ID NO. 6
MEFGLSWVFLVALFRGVQSQVTLKESGPVLVKPTETLTLTCTVSGFSLTSYGVHWIRQPPGKALEWL
AVIWAGGSISYSTSLMSRLTISKDTSKSQVVLTMTNMDPVDTATYYCARAYGDYVHYAMDYWGQGT
LVTVSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGGGGSGGGGSRVQPTESIVRFPNITNLCPFDEVFNATRFASVYAWNRKRISNCVADYSVLYNLAP
FFTFKCYGVSPTKLNDLCFTNVYADSFVIRGDEVRQIAPGQTGNIADYNYKLPDDFTGCVIAWNSNKL
DSKVSGNYNYLYRLFRKSNLKPFERDISTEIYQAGNKPCNGVAGFNCYFPLRSYSFRPTYGVGHQPY
RVVVLSFELLHAPATVCGPKKSGGGGSGGGGSILMQYIKANSKFIGIPMGLPQSIALSSLMVAQ▮
Signal peptide
Humanized 44H10 V_{H}
Human C_{H} (IgG₁)
Linkers
RBD (Omicron/B.1.1.529)
TpD
▮
Humanized (V19) LC-WuhanRBD
SEQ ID NO. 7
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVNF▮
Signal peptide
Humanized (V19) 44H10 V_{K}
Mutated residue
Human C_{K}
Linker
RBD (319-542)
▮
Humanized (V19) LC-WuhanRBD V2 (C-term trunc)
SEQ ID NO. 8
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKS▮
Signal peptide
Humanized (V19) 44H10 V_{K}
Mutated residue
Human C_{K}
Linker
RBD (319-531)
▮
Humanized (V19) LC-TpD
SEQ ID NO. 9
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSILMQYIKANSKFIGIPMGLPQSIALSSLMVAQ▮
Signal peptide
Humanized (V19) 44H10 V_{K}
Mutated residue
Human C_{K}
Linker
TpD
▮
Humanized (V19) LC-PADRE
SEQ ID NO. 10
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSAKFVAAWTLKAAA▮
Signal peptide
Humanized (V19) 44H10 V_{K}
Mutated residue
Human C_{K}
Linker
PADRE
▮
Humanized (V19) LC-PADRE-TpD
SEQ ID NO. 11
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSAKFVAAWTLKAAAPMGLPILMQYIKANSKFI
GIPMGLPQSIALSSLMVAQ▮
Signal peptide
Humanized (V19) 44H10 V_{K}
Mutated residue
Human C_{K}
Linker
PADRE
CatS cleavage site
TpD
▮
Humanized (V19) LC-OmicronRBD-TpD V2 (C-term trunc)
SEQ ID NO. 12
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFDEVFNA
TRFASVYAWNRKRISNCVADYSVLYNLAPFFTFKCYGVSPTKLNDLCFTNVYADSFVIRGDEVRQIAP
GQTGNIADYNYKLPDDFTGCVIAWNSNKLDSKVSGNYNYLYRLFRKSNLKPFERDISTEIYQAGNKPC
NGVAGFNCYFPLRSYSFRPTYGVGHQPYRVVVLSFELLHAPATVCGPKKSGGGGSGGGGSILMQYI
KANSKFIGIPMGLPQSIALSSLMVAQ▮
Signal peptide
Humanized (V19) 44H10 V_{K}
Mutated residue
Human C_{K}
Linkers
RBD (Omicron/B.1.1.529)
TpD
▮
Humanized (V19) LC-CoV1RBD
SEQ ID NO. 13
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSTNLCPFGEVFNATKFPSVYAWERKK
ISNCVADYSVLYNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCTPPALNCYWPL
NDYGFYTTTGIGYQPYRVVVLSFELLNAPATVCGPK▮
Signal peptide
Humanized (V19) 44H10 V_{K}
Mutated residue
Human C_{K}
Linker
SARS-CoV-1 RBD (320-510)
▮
Humanized (V19) LC-CoV1RBD-TpD
SEQ ID NO. 14
MEFGLSWVFLVALFRGVQSDIQMTQSPSSLSASVGDRVTITCRASQEISGYLTWLQQKPGKAPKLLI
YAASTLDSGVPSRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSTNLCPFGEVFNATKFPSVYAWERKK
ISNCVADYSVLYNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCTPPALNCYWPL
NDYGFYTTTGIGYQPYRVVVLSFELLNAPATVCGPKGGSILMQYIKANSKFIGIPMGLPQSIALSSLMV
AQ▮
Signal peptide
Humanized (V19) 44H10 V_{K}
Mutated residue
Human C_{K}
Linkers
SARS-CoV-1 RBD (320-510)
TpD
▮
Humanized (V20) LC-WuhanRBD
SEQ ID NO. 15
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVNF▮
Signal peptide
Humanized (V20) 44H10 V_{K}
Mutated residue
Human C_{K}
Linker
RBD (319-542)
▮
Humanized (V20) LC-WuhanRBD V2 (C-term trunc)
SEQ ID NO. 16
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKS▮
Signal peptide
Humanized (V20) 44H10 V_{K}
Mutated residue
Human C_{K}
Linker
RBD (319-531)
▮
Humanized (V20) LC-TpD
SEQ ID NO. 17
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSILMQYIKANSKFIGIPMGLPQSIALSSLMVAQ▮
Signal peptide
Humanized (V20) 44H10 V_{K}
Mutated residue
Human C_{K}
Linker
TpD
▮
Humanized (V20) LC-PADRE
SEQ ID NO. 18
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSAKFVAAWTLKAAA▮
Signal peptide
Humanized (V20) 44H10 V_{K}
Mutated residue
Human C_{K}
Linker
PADRE
▮
Humanized (V20) LC-PADRE-TpD
SEQ ID NO. 19
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSAKFVAAWTLKAAAPMGLPILMQYIKANSKFI
GIPMGLPQSIALSSLMVAQ▮
Signal peptide
Humanized (V20) 44H10 V_{K}
Mutated residue
Human C_{K}
Linker
PADRE
CatS cleavage site
TpD
▮
Humanized (V20) LC-OmicronRBD-TpD V2 (C-term trunc)
SEQ ID NO. 20
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFDEVFNA
TRFASVYAWNRKRISNCVADYSVLYNLAPFFTFKCYGVSPTKLNDLCFTNVYADSFVIRGDEVRQIAP
GQTGNIADYNYKLPDDFTGCVIAWNSNKLDSKVSGNYNYLYRLFRKSNLKPFERDISTEIYQAGNKPC
NGVAGFNCYFPLRSYSFRPTYGVGHQPYRVVVLSFELLHAPATVCGPKKSGGGGSGGGGSILMQYI
KANSKFIGIPMGLPQSIALSSLMVAQ▮
Signal peptide
Humanized (V20) 44H10 V_{K}
Mutated residue
Human C_{K}
Linkers
RBD (Omicron/B.1.1.529)
TpD
▮
Humanized (V20) LC-CoV1RBD
SEQ ID NO. 21
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSTNLCPFGEVFNATKFPSVYAWERKK
ISNCVADYSVLYNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCTPPALNCYWPL
NDYGFYTTTGIGYQPYRVVVLSFELLNAPATVCGPK▮
Signal peptide
Humanized (V20) 44H10 V_{K}
Mutated residue
Human C_{K}
Linker
SARS-CoV-1 RBD (320-510)
▮
Humanized (V20) LC-CoV1RBD-TpD
SEQ ID NO. 22
MEFGLSWVFLVALFRGVQSDIQMTQSPSSLSASVGDRVTITCRASQEISGYLTWLQQKPGKAPKLLI
YAASTLDSGVPKRFSGSGSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSTNLCPFGEVFNATKFPSVYAWERKK
ISNCVADYSVLYNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCTPPALNCYWPL
NDYGFYTTTGIGYQPYRVVVLSFELLNAPATVCGPKGGSILMQYIKANSKFIGIPMGLPQSIALSSLMV
AQ▮
Signal peptide
Humanized (V20) 44H10 V_{K}
Mutated residue
Human C_{K}
Linkers
SARS-CoV-1 RBD (320-510)
TpD
▮
Humanized (V21) LC-WuhanRBD
SEQ ID NO. 23
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVNF▮
Signal peptide
Humanized (V21) 44H10 V_{K}
Mutated residue
Human C_{K}
Linker
RBD (319-542)
▮
Humanized (V21) LC-WuhanRBD V2 (C-term trunc)
SEQ ID NO. 24
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFGEVFNA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKS▮
Signal peptide
Humanized (V21) 44H10 V_{K}
Mutated residue
Human C_{K}
Linker
RBD (319-531)
▮
Humanized (V21) LC-TpD
SEQ ID NO. 25
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSILMQYIKANSKFIGIPMGLPQSIALSSLMVAQ▮
Signal peptide
Humanized (V21) 44H10 V_{K}
Mutated residue
Human C_{K}
Linker
TpD
▮
Humanized (V21) LC-PADRE
SEQ ID NO. 26
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSAKFVAAWTLKAAA▮
Signal peptide
Humanized (V21) 44H10 V_{K}
Mutated residue
Human C_{K}
Linker
PADRE
▮
Humanized (V21) LC-PADRE-TpD
SEQ ID NO. 27
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGSGGSAKFVAAWTLKAAAPMGLPILMQYIKANSKFI
GIPMGLPQSIALSSLMVAQ▮
Signal peptide
Humanized (V21) 44H10 V_{K}
Mutated residue
Human C_{K}
Linker
PADRE
CatS cleavage site
TpD
▮
Humanized (V21) LC-OmicronRBD-TpD V2 (C-term trunc)
SEQ ID NO. 28
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSRVQPTESIVRFPNITNLCPFDEVFNA
TRFASVYAWNRKRISNCVADYSVLYNLAPFFTFKCYGVSPTKLNDLCFTNVYADSFVIRGDEVRQIAP
GQTGNIADYNYKLPDDFTGCVIAWNSNKLDSKVSGNYNYLYRLFRKSNLKPFERDISTEIYQAGNKPC
NGVAGFNCYFPLRSYSFRPTYGVGHQPYRVVVLSFELLHAPATVCGPKKSGGGGSGGGGSILMQYI
KANSKFIGIPMGLPQSIALSSLMVAQ▮
Signal peptide
Humanized (V21) 44H10 V_{K}
Mutated residue
Human C_{K}
Linkers
RBD (Omicron/B.1.1.529)
TpD
▮
Humanized (V21) LC-CoV1RBD
SEQ ID NO. 29
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSTNLCPFGEVFNATKFPSVYAWERKK
ISNCVADYSVLYNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCTPPALNCYWPL
NDYGFYTTTGIGYQPYRVVVLSFELLNAPATVCGPK▮
Signal peptide
Humanized (V21) 44H10 V_{K}
Mutated residue
Human C_{K}
Linker
SARS-CoV-1 RBD (320-510)
▮
Humanized (V21) LC-CoV1RBD-TpD
SEQ ID NO. 30
MEFGLSWVFLVALFRGVQSDIQMTQSPSSLSASVGDRVTITCRASQEISGYLTWLQQKPGKAPKLLI
YAASTLDSGVPKRFSGSRSGTDFTLTISSLQPEDFATYYCLQYTNYPLTFGQGTKLEIKRTVAAPSVFI
FPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGECGGGGSGGGGSTNLCPFGEVFNATKFPSVYAWERKK
ISNCVADYSVLYNSTFFSTFKCYGVSATKLNDLCFSNVYADSFVVKGDDVRQIAPGQTGVIADYNYKL
PDDFMGCVLAWNTRNIDATSTGNYNYKYRYLRHGKLRPFERDISNVPFSPDGKPCTPPALNCYWPL
NDYGFYTTTGIGYQPYRVVVLSFELLNAPATVCGPKGGSILMQYIKANSKFIGIPMGLPQSIALSSLMV
AQ▮
Signal peptide
Humanized (V21) 44H10 V_{K}
Mutated residue
Human C_{K}
Linkers
SARS-CoV-1 RBD (320-510)
TpD
▮

These different sequences can be modular, in the sense that any of the portions in the above sequences can be swapped with other analogous sequences or omitted or additional sequences can be included. For example, a TpD sequence may be used instead of or in addition to a PADRE sequence or they may be swapped from heavy chain to light chain and different linkers can be used. Similarly, it will be understood that the TpD or PADRE sequence may be replaced with an additional molecule, such as an antigen, that is the same or different from another such molecule bound elsewhere on the antibody. Typically, the antibody comprises at least one heavy chain and at least one light chain of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 listed above in any combination and, more typically, the antibody comprises or consists of two heavy and two light chains of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 listed above, in any combination.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (IIe or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

The polypeptides or antibodies of the present invention may also comprise additional sequences to aid in their expression, detection or purification. Any such sequences or tags known to those of skill in the art may be used. For example, and without wishing to be limiting, the antibodies may comprise a targeting or signal sequence (for example, but not limited to ompA), a detection tag, exemplary tag cassettes include Strep tag, or any variant thereof; see, e.g., U.S. Pat. No. 7,981,632, His tag, Flag tag having the sequence motif DYKDDDDK, Xpress tag, Avi tag, Calmodulin tag, Polyglutamate tag, HA tag, Myc tag, Nus tag, S tag, SBP tag, Softag 1, Softag 3, V5 tag, CREB-binding protein (CBP), glutathione S-transferase (GST), maltose binding protein (MBP), green fluorescent protein (GFP), Thioredoxin tag, or any combination thereof; a purification tag (for example, but not limited to a Hiss or His₆), or a combination thereof.

In another example, the additional sequence may be a biotin recognition site such as that described by Cronan et al in WO 95/04069 or Voges et al in WO/2004/076670. As is also known to those of skill in the art, linker sequences may be used in conjunction with the additional sequences or tags.

More specifically, a tag cassette may comprise an extracellular component that can specifically bind to an antibody with high affinity or avidity. Within a single chain fusion protein structure, a tag cassette may be located (a) immediately amino-terminal to a connector region, (b) interposed between and connecting linker modules, (c) immediately carboxy-terminal to a binding domain, (d) interposed between and connecting a binding domain (e.g., scFv) to an effector domain, (e) interposed between and connecting subunits of a binding domain, or (f) at the amino-terminus of a single chain fusion protein. In certain embodiments, one or more junction amino acids may be disposed between and connecting a tag cassette with a hydrophobic portion, or disposed between and connecting a tag cassette with a connector region, or disposed between and connecting a tag cassette with a linker module, or disposed between and connecting a tag cassette with a binding domain.

The antibodies may also be in a multivalent display. Multimerization may be achieved by any suitable method of known in the art. For example, and without wishing to be limiting in any manner, multimerization may be achieved using self-assembly molecules as described in Zhang et al (2004a; 2004b) and WO2003/046560.

Also encompassed herein are isolated or purified antibodies, polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

In other aspects, the antibodies may be linked to a cargo molecule; the antibodies may deliver the cargo molecule to a desired site and may be linked to the cargo molecule using any method known in the art (recombinant technology, chemical conjugation, chelation, etc.). The cargo molecule may be any type of molecule, such as a therapeutic or diagnostic agent. For example, and without wishing to be limiting in any manner, the therapeutic agent may be a radioisotope, which may be used for radioimmunotherapy; a toxin, such as an immunotoxin; a cytokine, such as an immunocytokine; a cytotoxin; an apoptosis inducer; an enzyme; or any other suitable therapeutic molecule known in the art. In the alternative, a diagnostic agent may include, but is by no means limited to a radioisotope, a paramagnetic label such as gadolinium or iron oxide, a fluorophore, a Near Infra-Red (NIR) fluorochrome or dye (such as Cy3, Cy5.5, Alexa680, Dylight680, or Dylight800), an affinity label (for example biotin, avidin, etc), fused to a detectable protein-based molecule, or any other suitable agent that may be detected by imaging methods. In a specific, non-limiting example, the antibody may be linked to a fluorescent agent such as FITC or may genetically be fused to the Enhanced Green Fluorescent Protein (EGFP).

The antibodies described herein specifically bind to their targets. Antibody specificity, which refers to selective recognition of an antibody for a particular epitope of an antigen, of the antibodies or fragments described herein can be determined based on affinity and/or avidity. Affinity, represented by the equilibrium constant for the dissociation of an antigen with an antibody (KD), measures the binding strength between an antigenic determinant (epitope) and an antibody binding site. Avidity is the measure of the strength of binding between an antibody with its antigen. Antibodies typically bind with a K_{D} of 10⁻⁵ to 10⁻¹¹ M. Any K_{D} greater than 10⁻⁴ M is generally considered to indicate non-specific binding. The lesser the value of the K_{D}, the stronger the binding strength between an antigenic determinant and the antibody binding site. In aspects, the antibodies described herein have a K_{D} of less than 10⁻⁴ M, 10⁻⁵ M, 10⁻⁶ M, 10⁻⁷ M, 10⁻⁸ M, or 10⁻⁹ M.

Also described herein are nucleic acid molecules encoding the antibodies and polypeptides described herein, as well as vectors comprising the nucleic acid molecules and host cells comprising the vectors.

Polynucleotides encoding the antibodies described herein include polynucleotides with nucleic acid sequences that are substantially the same as the nucleic acid sequences of the polynucleotides of the present invention. “Substantially the same” nucleic acid sequence is defined herein as a sequence with at least 70%, at least 75%, at least 80%, at least 85%, at least 90%, at least 91%, at least 92%, at least 93%, at least 94%, at least 95% identity to another nucleic acid sequence when the two sequences are optimally aligned (with appropriate nucleotide insertions or deletions) and compared to determine exact matches of nucleotides between the two sequences.

Suitable sources of DNAs that encode fragments of antibodies include any cell, such as hybridomas, that express the full-length antibody. The fragments may be used by themselves as antibody equivalents, or may be recombined into equivalents, as described above. The DNA deletions and recombinations described in this section may be carried out by known methods, such as those described in the published patent applications listed above in the section entitled “Functional Equivalents of Antibodies” and/or other standard recombinant DNA techniques, such as those described below. Another source of DNAs are single chain antibodies produced from a phage display library, as is known in the art.

The polynucleotides described herein may be used for example in vaccines, such as mRNA vaccines, as will be understood.

Additionally, the expression vectors are provided containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colEI, pCRI, pBR322, pMB9, pUC, pKSM, and RP4. Prokaryotic vectors also include derivatives of phage DNA such as MI3 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P J. Southern & P. Berg, J. Mol. Appl. Genet, 1:327-341 (1982); Subramani et al, Mol. Cell. Biol, 1:854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), all of which are incorporated by reference herein).

The expression vectors typically contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Also described herein are recombinant host cells containing the expression vectors previously described. The antibodies described herein can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, Chinese Hamster Ovary (CHO) cells, Baby Hamster Kidney (BHK) cells and many others. Suitable additional eukaryotic cells include yeast and other fungi. Useful prokaryotic hosts include, for example, E. coli, such as E. coli SG-936, E. coli HB 101, E. coli W3110, E. coli X1776, E. coli X2282, E. coli DHI, and E. coli MRC1, Pseudomonas, Bacillus, such as Bacillus subtilis, and Streptomyces.

These present recombinant host cells can be used to produce antibodies by culturing the cells under conditions permitting expression of the polypeptide and purifying the polypeptide from the host cell or medium surrounding the host cell. Targeting of the expressed polypeptide for secretion in the recombinant host cells can be facilitated by inserting a signal or secretory leader peptide-encoding sequence (See, Shokri et al, (2003) Appl Microbiol Biotechnol. 60 (6): 654-664, Nielsen et al, Prot. Eng., 10:1-6 (1997); von Heinje et al., Nucl. Acids Res., 14:4683-4690 (1986), all of which are incorporated by reference herein) at the 5′ end of the antibody-encoding gene of interest. These secretory leader peptide elements can be derived from either prokaryotic or eukaryotic sequences. Accordingly suitably, secretory leader peptides are used, being amino acids joined to the N-terminal end of a polypeptide to direct movement of the polypeptide out of the host cell cytosol and secretion into the medium.

The antibodies described herein can be fused to additional amino acid residues. Such amino acid residues can be a peptide tag to facilitate isolation, for example. Other amino acid residues for homing of the antibodies to specific organs or tissues are also contemplated.

In another aspect, described herein are methods of vaccinating subjects by administering a therapeutically effective amount of the antibodies or vaccines described herein to a mammal in need thereof, typically an adult, elderly, young, juvenile, or neonatal mammal. Therapeutically effective means an amount effective to produce the desired therapeutic effect, such as providing a protective immune response against the antigen in question.

Any suitable method or route can be used to administer the antibodies and vaccines described herein. Routes of administration include, for example, oral, intravenous, intraperitoneal, subcutaneous, or intramuscular administration.

It is understood that the antibodies described herein, where used in a mammal for the purpose of prophylaxis or treatment, will be typically administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection may, as is well known in the art, be formulated so as to provide quick, sustained or delayed release of the active ingredient after administration to the mammal.

Although human antibodies are particularly useful for administration to humans, they may be administered to other mammals as well. The term “mammal” as used herein is intended to include, but is not limited to, humans, laboratory animals, domestic pets and farm animals.

Also included herein are kits for vaccination, comprising a therapeutically or prophylactically effective amount of the antibodies described herein. The kits can further contain any suitable adjuvant for example or, in aspects, they exclude an adjuvant. Kits may include instructions.

Also described are methods of immunizing a subject against a disease or condition as well as methods of treating and/or preventing a disease or condition in a subject. Also described are related uses of the antibody or vaccine described herein for immunizing a subject against a disease or condition and/or for treating and/or preventing a disease or condition.

These methods and uses comprise administering the antibody or vaccine to a subject afflicted with the disease or condition, a subject suspected of being afflicted with the disease or condition, or a subject at risk of being afflicted with the disease or condition.

In aspects, the methods and uses further comprise administering a vaccine against tetanus and/or diphtheria toxoids to the subject and/or are carried out in a subject previously vaccinated against tetanus and/or diphtheria toxoids. Many examples of such vaccinations against tetanus and/or diphtheria toxoids exist, such as the Td Adsorbed vaccine available from Sanofi Pasteur. For example, if a potential vaccine recipient were immunized with Td one month prior to receiving a immunotargeted coronavirus vaccine which had TpD incorporated into the construct, the T-helper cells induced in the individual by the Td vaccine could act to significantly enhance the antibody response to the coronavirus antigens on the vaccine. This could provide an enhancement in terms of anti-coronavirus antibody responses in certain populations, such as the elderly, the immunocompromised, or other populations that may otherwise be poor responders.

In aspects, the vaccine against tetanus and/or diphtheria toxoids may administered to the subject substantially simultaneously with or prior to the vaccine, such as one or more days, weeks, months, or years prior to the vaccine, such as about one month prior to the vaccine.

While the vaccine may be use together with an adjuvant, it will be understood that, in typical aspects, the vaccine is administered without an adjuvant. In typical aspects, the vaccine is administered as a purified protein without an adjuvant.

The above disclosure generally describes the present invention. A more complete understanding can be obtained by reference to the following specific examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein

Without further description, it is believed that one of ordinary skill in the art can, using the preceding description and the following illustrative examples, make and utilize the compounds of the present invention and practice the claimed methods. The following working examples therefore, specifically point out the typical aspects of the present invention, and are not to be construed as limiting in any way the remainder of the disclosure.

### EXAMPLES

### Example 1: This Example Illustrates the Co-Transfection of Plasmids Encoding Antibody Heavy and Light Chains into Freestyle 293-F Cells for the Expression of Immunotargeting mAbs

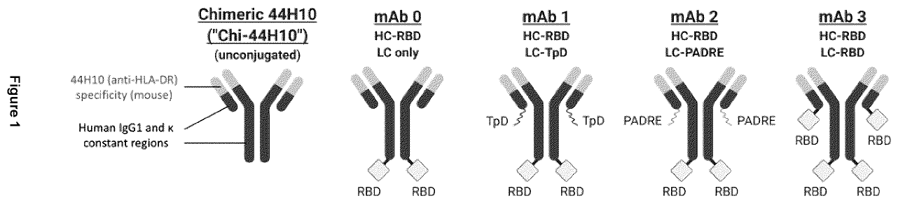

FIG. 1 shows schematics of the unconjugated chimeric human IgG1 anti-HLA-DR 44H10 (mouse V_{H} and V_{L}) antibody (“Chi-44H10”) and four different immunotargeting mAbs with SARS-CoV-2 Spike protein Receptor Binding Domain (RBD) and universal T-helper determinants (TpD or PADRE) integrated into either immunoglobulin heavy (HC) or light (LC) chains as indicated (44H10 as described by Dubiski et al. (1988) 1°).

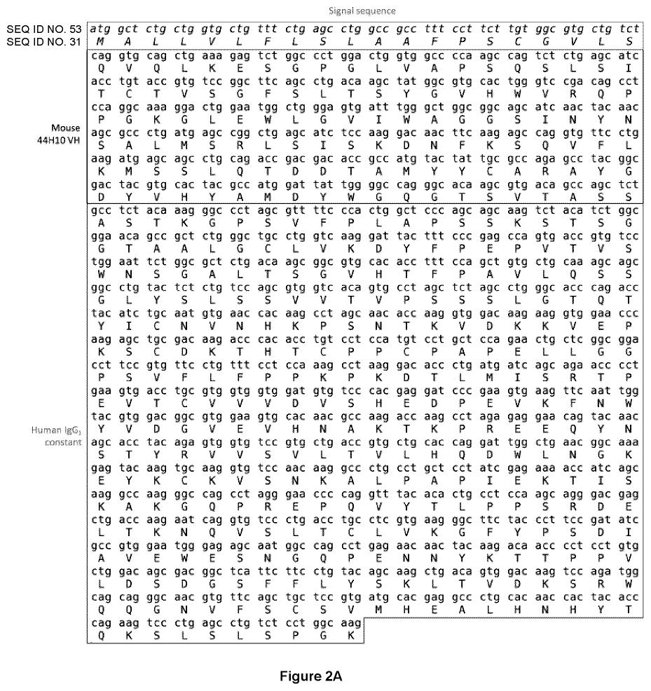

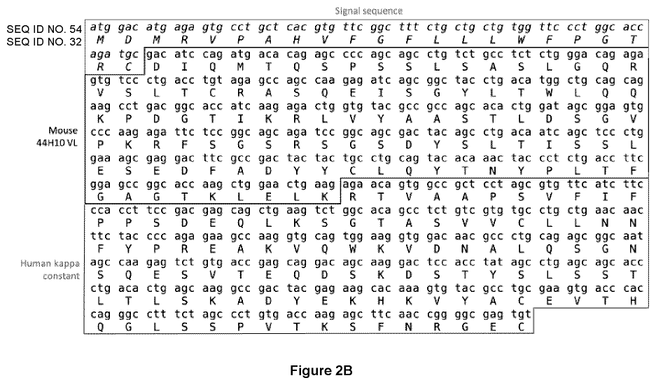

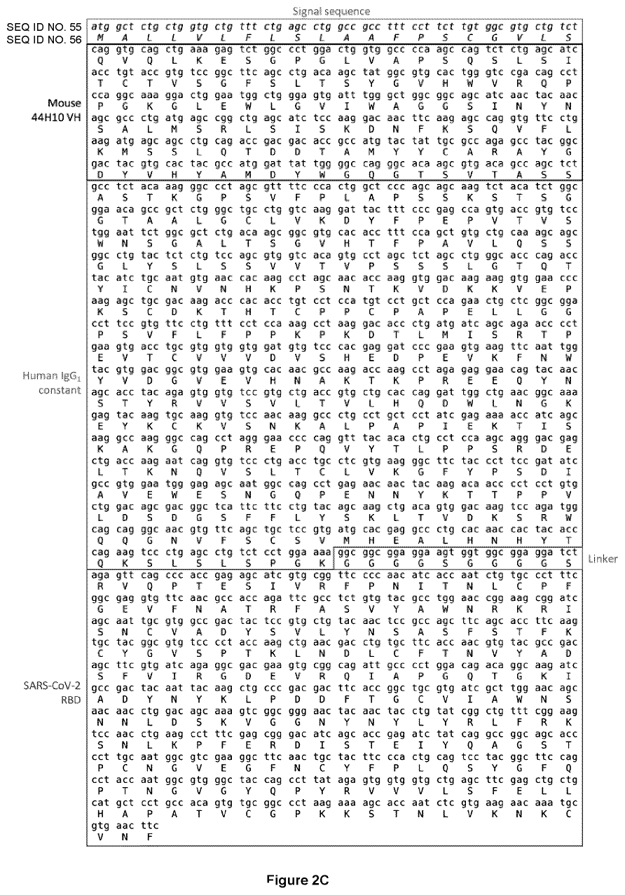

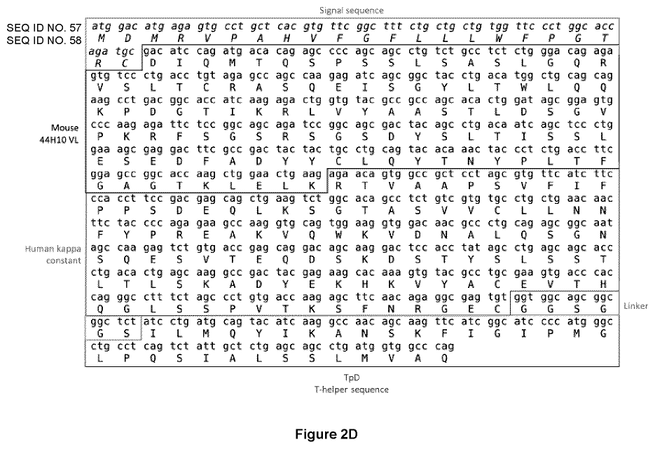

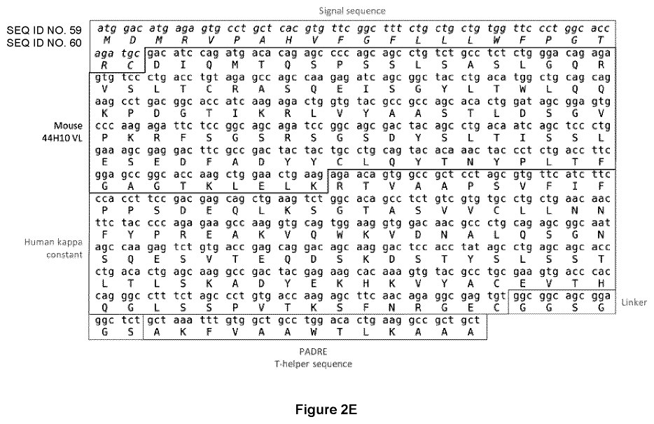

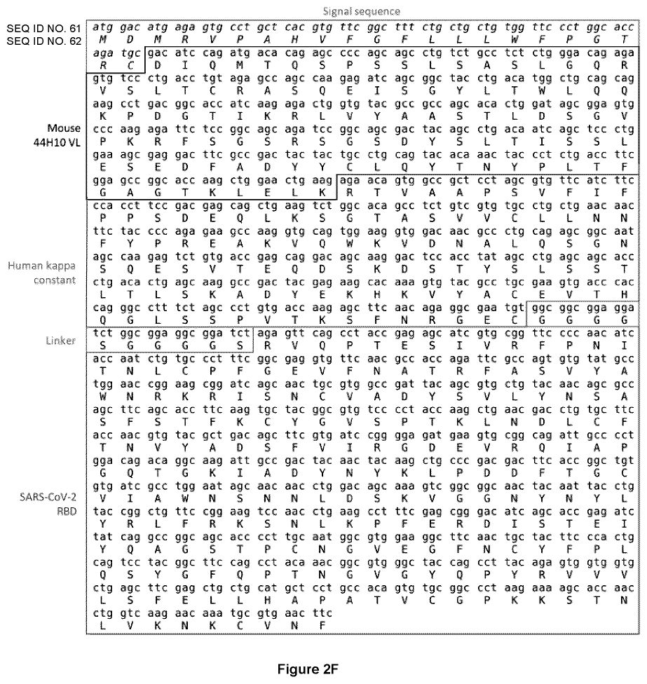

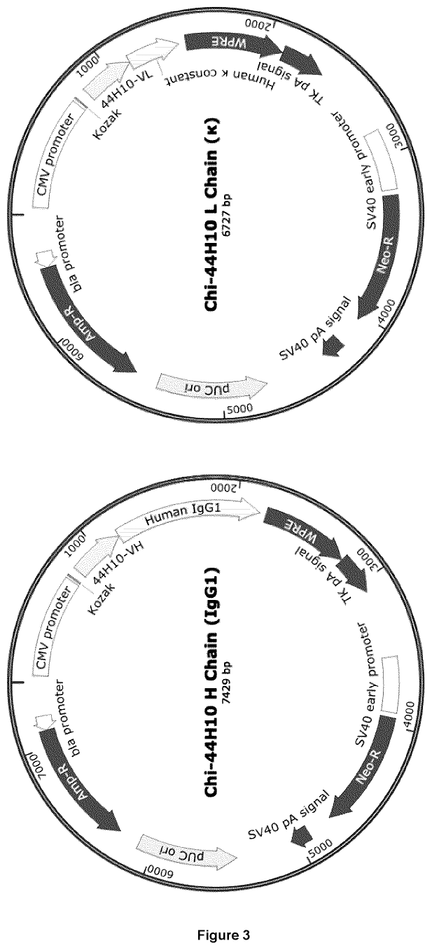

DNA plasmids encoding heavy and light chain antibody constructs were designed in the pcDNA3.4 TOPO expression vector and optimized for Homo sapiens expression using GeneArt Gene Synthesis (Invitrogen) (FIGS. 2 and 3). These constructs were maxiprepped using PureLink HiPure Plasmid Maxiprep Kits (Invitrogen).

FreeStyle 293-F Cells were cultured in 500 mL polycarbonate Erlenmeyer flasks (Triforest Labware) in FreeStyle 293 Expression Medium (Gibco) and split to a density of 0.8×10⁶ cells/ml at least one hour before transfection. Cells were transfected using the FectoPRO Reagent (Polyplus) following manufacturer instructions at a 1:1 DNA to FectoPRO ratio. 90 μg of plasmid DNA was used for transfection (60 μg of heavy chain DNA and 30 μg of light chain DNA) for every 200 ml of cell culture. Transfected cells were incubated in a 37° C., 5% CO₂ shaking incubator for 5 to 7 days to allow for the expression and self-assembly of heavy and light chain gene products.

### Example 2. This Example Illustrates the Purification of Immunotargeting mAbs from the Supernatant of Transfected FreeStyle 293-F Cells

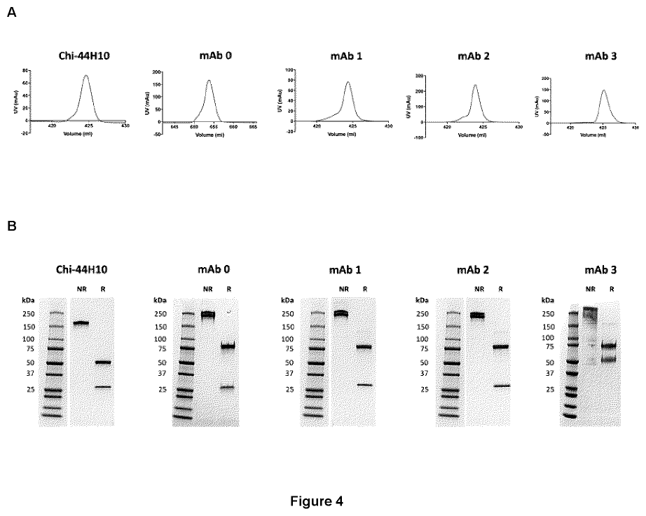

Transfected cell culture supernatants were collected and filtered through 0.22 μM Steritop filters (Millipore Sigma) before loading onto protein A affinity columns using the ÄKTA start protein purification system (Cytiva Life Sciences). Following loading, samples were washed with 1× phosphate-buffered saline (PBS) then eluted with 100 mM glycine pH 2.2 and immediately neutralized with 1 M Tris pH 9. Samples collected from the elution peaks (FIG. 4A) were buffer exchanged into PBS using PD-10 desalting columns (Cytiva Life Sciences) and adjusted to a final concentration of 1 mg/ml using Nanodrop 2000 Spectrophotometer measurements (Thermo Scientific).

Samples were run on 10-well 4-20% SDS-PAGE gradient gels in non-reducing and reducing sample buffer (±2-Mercaptoethanol) at 250 V for 20 minutes (Bio-Rad). Gels were stained with Coomassie Brilliant Blue for protein visualization (FIG. 4B). A band corresponding to the intact Chi-44H10 antibody is detected in the non-reducing condition at 150 kDa, and individual heavy and light chain bands in the reducing condition are detected at 50 kDa and 25 kDa, respectively. Bands for the immunotargeting mAbs bearing heavy and/or light chain conjugates are shifted to increased molecular weights relative to the unmodified Chi-44H10 bands in accordance with the size of the added entity.

### Example 3. This Example Illustrates the Procedure for the Fluorescent Labeling of Unconjugated and Conjugated Antibodies for Subsequent Flow Cytometric Experiments

Purified antibodies were diluted at a concentration of 100 UM in PBS and incubated in a 10-fold molar excess of TCEP at room temperature for 30 minutes to reduce disulfide bonds and render cysteines accessible for labeling. After 30 minutes, Alexa Fluor C₅ Maleimide dye (Invitrogen) was added to each reaction at a concentration of 10 mM for a 10-20-molar excess of dye to protein. Samples were incubated overnight at 4° C. protected from light. Free, unconjugated dye was washed out of solution using PBS and Amicon 30K Ultra-0.5 mL Centrifugal Filters (Millipore Sigma) and the concentration of labeled protein was assessed by Nanodrop measurement.

### Example 4. This Example Illustrates the Flow Cytometric Procedure for the Assessment of Fluorescently Labeled Immunotargeting mAb Binding to the B-Lymphoblastoid Cell Line BJAB

B lymphoblastoid BJAB cells (Thermo Scientific) (described in Menezes et al. (1975) 11) were grown in supplemented RPMI medium containing 10% fetal bovine serum (FBS) in a 37° C., 5% CO₂ incubator. Cells were collected in a 15 ml conical tube and centrifuged at 300 g for 5 minutes. Cell pellets were resuspended in staining buffer (PBS containing 2% FBS and 0.05% NaN₃) at a concentration of 1×10⁶ cells/ml, and 200 μl of the cell suspension was dispensed into the wells of a polystyrene, V-bottom 96-well plate (Greiner Bio-One) for staining. The plate was centrifuged at 300 g for 5 minutes, and the cells were resuspended in an Fc-block solution and incubated at 4° C. for 30 minutes. Cells were washed once in staining buffer to remove the Fc block solution. The cells were resuspended in 50 μl of 0.1 μg/μl pre-labeled (A488) antibody (per described in Example 3) and incubated at 4° C. for 1 hour. The cells were washed twice in staining buffer, then resuspended in staining buffer containing 0.5 μM DAPI (PromoCell) for the exclusion of dead cells and debris. A control consisting of cells incubated with a labeled, unrelated isotype-matched antibody served as a negative control in this experiment.

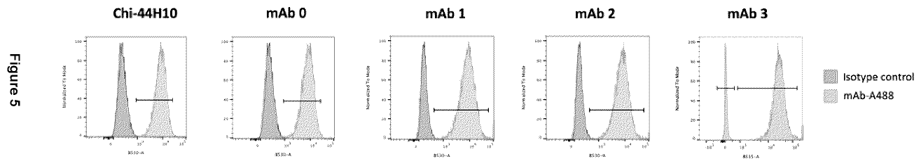

The binding of fluorescent anti-HLA-DR antibodies to BJAB cells was analyzed in a BD LSR II cytometer in the B530 channel. Gates on the histograms represent the positive signal established by the positive control (anti-CD19 antibody Denintuzumab). The binding of fluorescently labeled Chi-44H10 and immunotargeting mAbs to BJAB cells is depicted in FIG. 5, validating the ability of these antibodies to target HLA-DR expressed on B-lymphoblastoid cells.

### Example 5. This Example Illustrates the Flow Cytometric Procedure for the Assessment of RBD Structural Integrity on Immunotargeting mAbs Using Fluorescently Labeled Anti-RBD Antibodies

The structural integrity of the SARS-CoV-2 RBD attachment on the immunotargeting mAbs was assessed by the same technique described in Example 4, using three antibodies targeting distinct sites of the RBD as conformational probes: CR3022^{12,13}, S309¹⁴ and VHH72¹⁵. For this analysis, BJAB cells were first incubated with 50 ul of 0.1 ug/ul unlabeled purified Chi-44H10 or immunotargeting mAbs, washed once, then stained with 50 ul of 0.1 μg/ul pre-labeled (A488) CR3022, S309 and VHH72-Fc antibodies. A set of cells only incubated with labelled anti-RBD antibodies in the absence of immunotargeting mAbs served as a negative control.

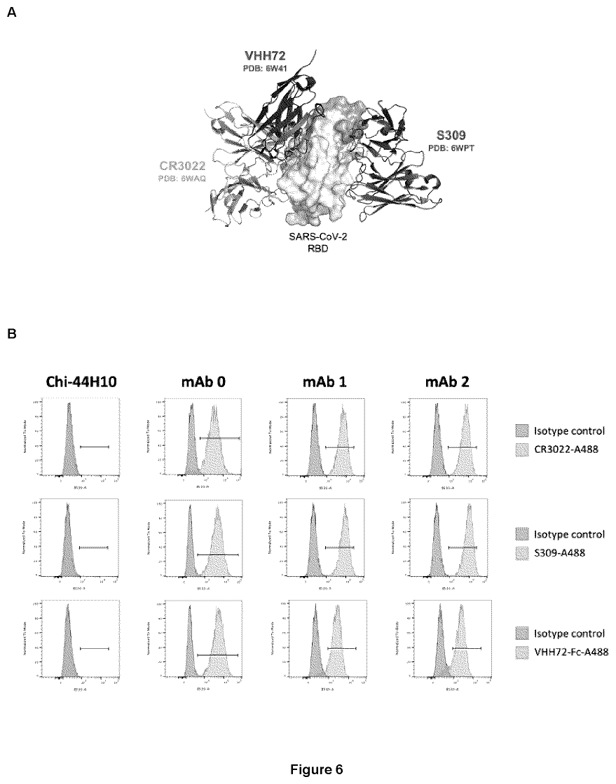

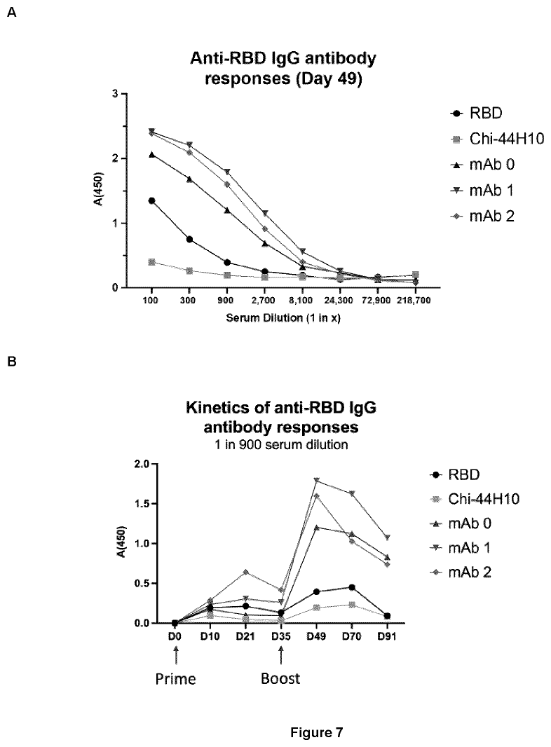

The binding of fluorescently labeled anti-RBD antibodies to immunotargeting mAbs bound to BJAB cells, but not direct binding to BJAB cells, is depicted in FIG. 6. This indicates that RBD epitopes on the immunotargeting mAbs are structurally intact and accessible. No anti-RBD antibody binding to unmodified Chi-44H10 was detected, as this antibody lacks any RBD.

### Example 6. This Example Illustrates the Procedure for the Immunization of Animals with Immunotargeting mAbs

Immunogens were diluted to a concentration of 0.05 mg/ml in PBS. Female New Zealand white rabbits (5 per group) were immunized by Cedarlane Laboratories with 50 ug of unadjuvanted immunogen via subcutaneous or intramuscular injection, followed by a boost of the same dose at 5 weeks post-prime (D35). A control group immunized with soluble RBD was used to examine the specific effect of immunotargeting by anti-HLA-DR antibody conjugation, using a dose corresponding to an equimolar amount of RBD compared to the immunotargeting mAbs (i.e. 7.5 μg).

Rabbits were bled before immunization (DO) and at days 10, 21, 35, 49, 70 and 92 post-primary immunization (Table 1).

Table 1 (immediately below) outlines the dosage and schedule set forth for rabbit immunizations with soluble RBD (sRBD), Chi-44H10 or immunotargeting mAbs. For subcutaneous administration, animals were injected with ¼ of the total dose at 4 separate sites for both the prime and boost immunizations. For intramuscular administration, animals were injected with the total dose at a single site for both the prime and boost immunizations. Note that the selected dose of 7.5 μg of sRBD is equimolar to the amount of RBD in 50 μg of each immunotargeting antibody.

| | |
|---|---|
| Day 0 | Pre-immune bleed |
| | Primary immunization (50 ug of antibody or 7.5 ug of RBD) |
| Day 10 | Bleed 1 |
| Day 21 | Bleed 2 |
| Day 35 | Bleed 3 |
| | Booster immunization (50 ug of antibody or 7.5 ug of RBD) |
| Day 49 | Bleed 4 |
| Day 70 | Bleed 5 |
| Day 91 | Bleed 6 |

### Example 7. This Example Illustrates the Procedure for the Collection of Blood from Rabbits and Preparation of Serum for Subsequent Assays

The following procedures were performed at Cedarlane Laboratories in Burlington, ON, Canada. Blood was collected into red top vacutainer tubes and incubated at 3-4 hours at room temperature to allow for clotting. The tubes were centrifuged at 4° C. at ˜3000 rpm for 20 minutes to pellet the red blood cells and debris. Supernatants were poured off into appropriate tubes and stored at −20° C. before shipping on dry ice.

### Example 8. This Example Illustrates the ELISA Procedure for the Assessment of Serum Anti-Sars-CoV-2 RBD Elicited by Immunization with Immunotargeting mAbs

Immulon 4 HBX ELISA plates (Thermo Scientific) were coated with 50 μl of 2 μg/mL SARS-CoV-2 RBD diluted in PBS and incubated overnight at 4° C. The coating solution was removed and plates were washed three times with PBS-T (PBS containing 0.1% Tween). Plates were incubated with blocking buffer (PBS-T containing 3% non-fat milk) for 1 hour at room temperature. Serum samples were serially pre-diluted in diluent buffer (PBS-T containing 1% non-fat milk) in 1.2 mL microtiter dilution tubes (Thermo Fisher). The blocking solution was discarded and 100 μl of pre-diluted sera was added to the plates and incubated at room temperature for 2 hours. Plates were washed three times with PBS-T and incubated with Goat Anti-Rabbit IgG H&L (HRP) secondary antibody (Abcam 97051) at a 1:10,000 dilution for 1 hour at room temperature. Plates were developed using a TMB Substrate Reagent Set (BD) following manufacturer instructions; reactions were stopped at 10 minutes by the addition of 2N HCl. Plates were read at an absorbance of 450 nm using a Synergy Neo2 Multi-Mode Assay Microplate Reader (Biotek Instruments).

### Example 9. This Example Illustrates the Procedure for the Pseudovirus Neutralization Assay Used to Assess Neutralization Potency of Antibodies Elicited by Immunization with Immunotargeting mAbs

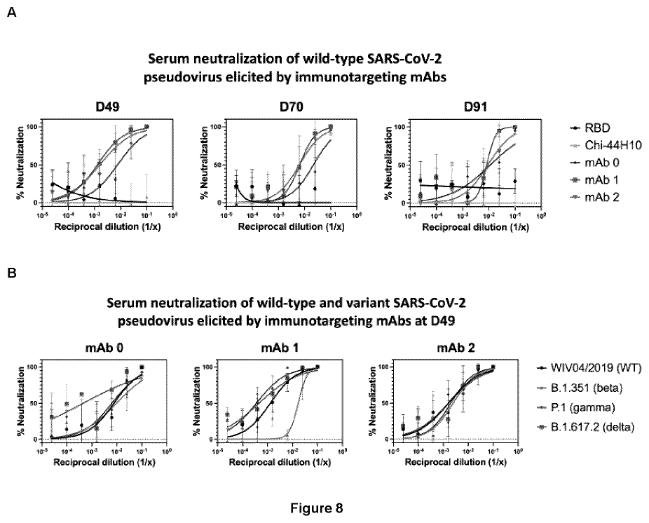

The procedure described in this Example was adapted from Crawford et al. (2020)¹⁶. 293T cells were co-transfected with a lentiviral backbone encoding the luciferase reporter gene (BEI NR52516), a plasmid expressing the SARS-CoV-2 Spike (BEI NR52310) and plasmids encoding the HIV structural and regulatory proteins Tat (BEI NR52518), Gag-pol (BEI NR52517) and Rev (BEI NR52519). Co-transfection of the five plasmids was performed using BioT reagent (Bioland Scientifics) following manufacturer instructions. 24 hours post-transfection at 37° C., the media was supplemented with 5 mM sodium butyrate (NaB) and the cells were further incubated for an additional 24 hours at 30° C. prior to pseudovirus (PsV) harvesting. The neutralization assay was performed using 293T-ACE2 cells (BEI NR52511) as previously described¹⁶ with few modifications. Briefly, rabbit sera was inactivated by 30-minute incubation at 56° C. 4-fold serial dilutions of the inactivated sera were incubated for 1 hour at 37° C. with SARS-CoV-2 PsV and subsequently added to 293T-ACE2 cells (BEI NR52511) seeded in Poly-L-lysine (Sigma-Aldrich) coated plates 24 hours prior to the experiment. After 48 hours of incubation, PsV neutralization was monitored adding 50 μl of Britelite plus reagent (PerkinElmer) to 50 ul of cells. After 2 minutes of incubation, the volume was transferred to a 96-well white plate (Sigma-Aldrich) to measure luminescence in relative light units (RLUs) using a Synergy Neo2 Multi-Mode Assay Microplate Reader (Biotek Instruments). The data were analyzed by non-linear regression (FIG. 8, Tables 2 and 3). It has now been established that the best correlate of Covid-19 vaccine efficacy in humans is the presence of virus-neutralizing antibodies in the serum of vaccine recipients (17). Therefore the virus-neutralizing antibody responses described in Example 9 (FIGS. 8 & 9, Tables 2, 3 & 4) represent a clear indication of the vaccine properties of the modified antibody molecules for SARS-CoV-2.

Table 2 (immediately below) outlines the half maximal Inhibitory Concentration (IC50) values for the pseudovirus neutralization data shown in FIG. 8A, indicating the serum dilution at which 50% of wild-type pseudovirus is neutralized in the assay. Note that no pseudovirus neutralization was detected in the pre-boost (D0, D10, D21 and D35) serum of any immunization group.

| | D 49 | D 70 | D 91 |
|---|---|---|---|
| SRBD | — | — | — |
| Chi-44H10 | — | — | — |
| mAb 0 | 1:130 | 1:45 | 1:88 |
| mAb 1 | 1:778 | 1:153 | 1:120 |
| mAb 2 | 1:627 | 1:157 | 1:108 |

Table 3 (immediately below) outlines the half maximal Inhibitory Concentration (IC50) values for the pseudovirus neutralization data shown in FIG. 8B, indicating the serum dilution at which 50% of pseudovirus of the specified strain is neutralized at D49 post-primary immunization.

| | WIV04/2019 | B.1.351 | P.1 | B.1.617.2 |
|---|---|---|---|---|
| | Wild-type | Beta variant | Gamma variant | Delta variant |
| mAb 0 | 1:130 | 1:103 | 1:158 | 1:2227 |
| mAb 1 | 1:778 | 1:59 | 1:2807 | 1:2212 |
| mAb 2 | 1:627 | 1:401 | 1:501 | 1:617 |

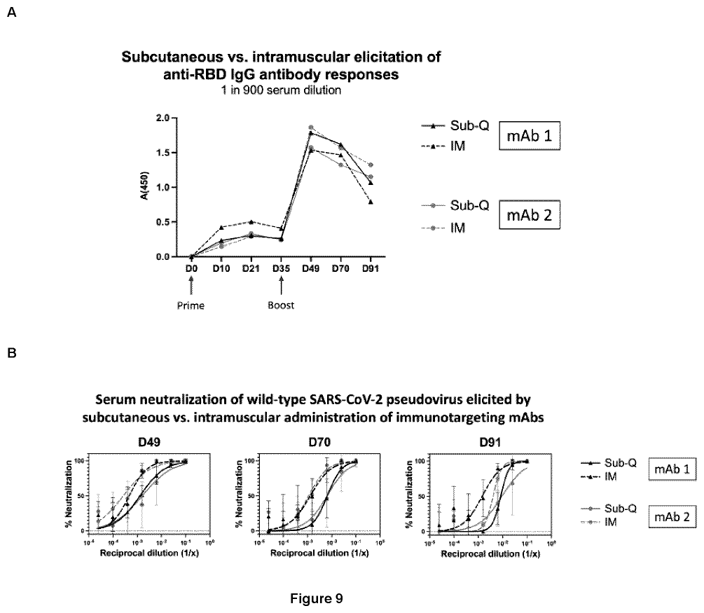

Table 4 (immediately below) outlines the half maximal Inhibitory Concentration (IC50) values for the pseudovirus neutralization data shown in FIG. 9B comparing subcutaneous (sub-Q) and intramuscular (IM) administration routes, indicating the serum dilution at which 50% of wild-type pseudovirus is neutralized in the assay. Note that no pseudovirus neutralization was detected in the pre-boost (D0, D10, D21 and D35) serum of any immunization group.

| | | D 49 | D 70 | D 91 |
|---|---|---|---|---|
| mAb 1 | Sub-Q | 1:778 | 1:153 | 1:120 |
| | IM | 1:2173 | 1:778 | 1:706 |
| mAb 2 | Sub-Q | 1:627 | 1:157 | 1:109 |
| | IM | 1:3832 | 1:905 | 1:249 |

### REFERENCES

1. Janeway, C. A. How the immune system protects the host from infection. Microbes and Infection vol. 3 1167-1171 (2001).
2 Petrovsky, N. Comparative Safety of Vaccine Adjuvants: A Summary of Current Evidence and Future Needs. Drug Safety vol. 38 1059-1074 (2015).
3. Carayanniotis, G. & Barber, B. H. Adjuvant-free IgG responses induced with antigen coupled to antibodies against class II MHC. Nature 327, 59-61 (1987).
4. Barber, B. H. The immunotargeting approach to adjuvant-independent subunit vaccine design. Semin. Immunol. 9, 293-301 (1997).
5. Keler, T., He, L., Ramakrishna, V. & Champion, B. Antibody-targeted vaccines. Oncogene 26, 3758-3767 (2007).
6. Gil, F. et al. Targeting antigens to an invariant epitope of the MHC Class II DR molecule potentiates the immune response to subunit vaccines. Virus Res. 155, 55-60 (2011).
7. Knutson, K. L. & Disis, M. L. Tumor antigen-specific T helper cells in cancer immunity and immunotherapy. Cancer Immunology, Immunotherapy vol. 54 721-728 (2005).
8. Alexander, J. et al. Development of high potency universal DR-restricted helper epitopes by modification of high affinity DR-blocking peptides. Immunity 1, 751-761 (1994).
9. Fraser, C. C. et al. Generation of a universal CD4 memory T cell recall peptide effective in humans, mice and non-human primates. Vaccine 32, 2896-2903 (2014).
10. Dubiski, S., Cinader, B., chou, C. T., Charpentier, L. & Letarte, M. Cross-reaction of a monoclonal antibody to human MHC class II molecules with rabbit B cells. Mol. Immunol. 25, 713-718 (1988).
11. Menezes, J., Leibold, W., Klein, G. & Clements, G. Establishment and characterization of an Epstein-Barr virus (EBC)-negative lymphoblastoid B cell line (BJA-B) from an exceptional, EBV-genome-negative African Burkitt's lymphoma. Biomedicine 22, 276-284 (1975).
12. Yuan, M. et al. A highly conserved cryptic epitope in the receptor binding domains of SARS-CoV-2 and SARS-CoV. Science (80−.). 368, 630-633 (2020).
13. Tian, X. et al. Potent binding of 2019 novel coronavirus spike protein by a SARS coronavirus-specific human monoclonal antibody. Emerging Microbes and Infections vol. 9 382-385 (2020).
14. Pinto, D. et al. Cross-neutralization of SARS-CoV-2 by a human monoclonal SARS-CoV antibody. Nature (2020) doi: 10.1038/s41586-020-2349-y.
15. Wrapp, D. et al. Structural Basis for Potent Neutralization of Betacoronaviruses by Single-Domain Camelid Antibodies. Cell 181, 1004-1015.e15 (2020).
16. Crawford, K. H. et al. Protocol and reagents for pseudotyping lentiviral particles with SARS-CoV-2 Spike protein for neutralization assays. bioRxiv 2020.04.20.051219 (2020) doi: 10.1101/2020.04.20.051219.
17. Earle, K. A. et al. Evidence for antibody as a protective correlate for COVID-19 vaccines. Vaccine 39, 4423 (2021).

### Example 10. This Example Illustrates the Procedure for Humanization of the Mouse mAb 44H10 Targeting HLA-DR

### Abstract

The ongoing Coronavirus Disease 2019 (COVID-19) pandemic, caused by the coronavirus designated SARS-CoV-2, has thus far (by April 2022) infected nearly 500 million people, resulting in greater than 6 million deaths worldwide. A safe and effective vaccine which provides durable protection against COVID-19 is thus required for long-term control of the virus. The ability to specifically deliver cargo to Antigen-Presenting Cells (APCs) and B cells through a pan-reactive anti-HLA-DR targeting monoclonal antibody (mAb) offers broad potential for next-generation immunointervention across human health indications. This example describes in molecular detail the unexpected path to humanization of the mouse mAb 44H10 targeting HLA-DR, made possible by molecular insights derived from a co-crystal structure of the 44H10 Fab-HLA-DR complex and subsequent structure-based design.

### Materials and Methods

### Design, Expression and Purification of Recombinant Antibodies

DNA plasmids encoding heavy and light chain antibody constructs were designed in the pcDNA3.4 TOPO mammalian expression vector and optimized for Homo sapiens expression at GeneArt (Invitrogen). These constructs were maxiprepped using PureLink HiPure Plasmid Maxiprep Kits (Invitrogen). FreeStyle 293-F Cells were cultured in 500 mL polycarbonate Erlenmeyer flasks (Triforest Labware) in FreeStyle 293 Expression Medium (Gibco) and split to a density of 0.8×10⁶ cells/ml at least one hour before transfection. Cells were transfected using the FectoPRO Reagent (Polyplus) following manufacturer instructions at a 1:1 DNA to FectoPRO ratio. 90 ug of plasmid DNA was used for transfection (60 ug of heavy chain DNA and 30 ug of light chain DNA) for every 200 ml of cell culture. Transfected cells were incubated in a 37° C., 5% CO₂ shaking incubator for 5 to 7 days to allow for the expression and self-assembly of heavy and light chain gene products.

Transfected cell culture supernatants were collected and filtered through 0.22 μM Steritop filters (Millipore Sigma) before loading onto protein A affinity columns using the ÄKTA start protein purification system (Cytiva Life Sciences). Following loading, samples were washed with 1× phosphate-buffered saline (PBS) then eluted with 100 mM glycine, pH 2.2 and immediately neutralized with 1 M Tris, pH 9. Samples collected from the elution peaks were buffer exchanged into PBS using Amicon 30K Ultra-0.5 mL Centrifugal Filters (Millipore Sigma) and frozen at −80° C. until use.

Chimeric 44H10 Fab for crystallization was transfected as described above and purified by KappaSelect affinity chromatography, followed by MonoS ion exchange chromatography using 20 mM NaOAc, pH 5.6+1M KCl, and finally size exclusion chromatography on a Superdex 200 Increase 10/300 GL in TBS, pH 8 (all from Cytiva).

### Design, Expression and Purification of Recombinant HLA-DR

The extracellular domains of HLA-DR (DRA*01: 01/DRB1*04:01) α and β chains were designed in pcDNA3.4 TOPO, as described above. Both chains contained C-terminal TEV-cleavable fos/jun zippers to promote dimerization and 6×His tags for purification purposes⁶. The HLA-DR molecule was expressed with the Influenza Hemagglutinin (HA) peptide covalently attached via a flexible linker to the N terminus of the β chain. α and β chain plasmids were co-transfected in a 1:1 ratio into HEK 293S (GnT |^{-/-}) cells. Recombinant HLA-DR was purified by affinity chromatography via a His Trap Ni-NTA column (Cytiva) and eluted using 20 mM Tris, pH 8.0 containing 500 mM imidazole. Subsequent size exclusion chromatography was performed in TBS, pH 8 on a Superdex 200 Increase 10/300 GL. The purified HLA-DR was subjected to overnight treatment with EndoH and TEV proteases at ratios of 5:1 and 20:1, respectively, for deglycosylation and cleavage of the fos/jun zipper. A second round of affinity and size exclusion purification was performed on the cleaved HLA-DR before complexation with chimeric 44H10 Fab for crystallization trials.

### Flow Cytometric Analysis of Recombinant Antibody Binding to BJAB Cells

B lymphoblastoid BJAB cells (Thermo Scientific), known to have a high level of HLA-DR expression (described in Menezes et al. (1975)²), were grown in supplemented RPMI medium containing 10% fetal bovine serum (FBS) in a 37° C., 5% CO₂ incubator. Cells were collected in a conical tube and centrifuged at 300 g for 5 min. Cell pellets were resuspended in staining buffer (PBS containing 2% FBS and 0.05% NaN₃) at a concentration of 1×10⁶ cells/ml, and 200 μl of the cell suspension was dispensed into the wells of a polystyrene, V-bottom 96-well plate (Greiner Bio-One) for staining. The plate was centrifuged at 300 g for 5 min, and the cells were incubated in Fc Block (BD Biosciences) for 10 min at room temperature. Parental or humanized 44H10 mAb were then added to the cells at the specified concentrations and left to incubate for 1 h at 4° C. Cells were washed twice, then stained with Alexa Fluor 488 AffiniPure Goat Anti-Human IgG, Fcγ fragment specific (1:400 dilution, Jackson ImmunoResearch) for 30 min at 4° C. After two additional washes, cells were resuspended in a 1/20 dilution of propidium iodide (Thermo Scientific) for the exclusion of dead cells and debris. An isotype-matched antibody incapable of binding BJAB cells served as a negative control in this experiment. Binding signals were measured using a BD LSR II cytometer in the B515 channel using the BD FACSDiva™ Software, and further analyzed using the FlowJo™ Software.

### Crystallization and Structure Determination

Chimeric 44H10 Fab was mixed with HLA-DR in a 1.5-molar excess, and excess Fab was purified away via size exclusion chromatography (Superdex 200 Increase 10/300 GL, Cytiva). The protein complex was concentrated to 8 mg/ml and mixed with a mother liquor of 1.75 M ammonium sulfate, 0.1 M sodium cacodylate, pH 7.1 and 0.2 M sodium chloride, as well as with crystal seeds previously obtained in a condition of 2 M ammonium sulfate and 0.2 M bis-tris pH 5.5, in a ratio of 2:1:3 (protein: seed: mother liquor). Crystals appeared after ˜12 days and grew steadily until day ˜32, at which time they were cryprotected in 15% (v/v) ethylene glycol before being flash-frozen in liquid nitrogen. Data were collected at the 23-ID-D beamline at the Argonne National Laboratory Advanced Photon Source. Datasets were processed, merged and scaled using XDS⁷. The structure were determined by molecular replacement using Phaser⁸. Refinement of the structures was performed using phenix.refine⁹ and iterations of refinement using Coot¹⁰. Access to all software was supported through SBGrid¹¹.

### Biolayer Interferometry

Real-time analysis of binding kinetics was measured using the Octet RED96 BLI system (Sartorius). Parental or humanized 44H10 antibodies were immobilized onto Protein A biosensors (Sartorius) at a concentration of 10 μg/mL until a threshold response of 0.7 nm. Baseline, association, and dissociation steps were conducted at 25° C. for 180 s in kinetics buffer (PBS, PH 7.4, 0.01% BSA, and 0.002% Tween). Baseline readings were established in buffer-containing wells and association events were measured by dipping loaded biosensors into wells containing a serial dilution of HLA-DR, starting at 500 nM. Dissociation was subsequently measured by transfer of biosensors back into buffer-containing wells. Biosensors were regenerated using 100 mM glycine, pH 2.2. Kinetics data were analyzed using the FortéBio Octet Data Analysis software 9.0.0.6, and curves were fitted to a 1:1 binding model.

### Results/Discussion

Antibody humanization is a method used to reduce the amount of foreign content in antibodies generated from immunized hosts, such as rodents and rabbits. The ultimate goal of humanization is to prevent unwanted immunogenicity against the molecule in humans, while retaining the affinity and specificity of the parental non-human antibody. Described herein is an approach used to humanize a previously disclosed parental antibody, 44H10¹, for the immunotargeting of HLA-DR-expressing cells in humans.

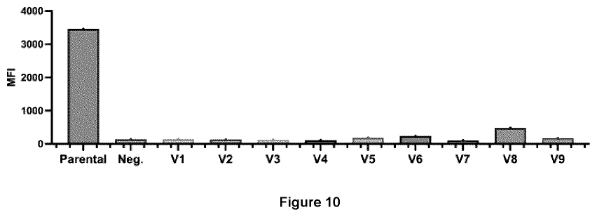

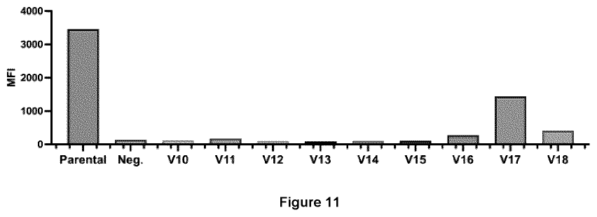

Humanized 44H10 variants were generated by CDR grafting. Using the IMGT database, the murine 44H10 VH and VL regions were aligned to all human VH and VL germline frameworks. The closest related VH and VL framework regions were selected as templates for CDR grafting based on the IMGT CDR definition. In total, 3 VH and 3 VL humanized variable regions were synthesized (Table 5) and each VH/VL combination pair was generated for screening, resulting in 9 humanized variants denoted as V1-V9 (Table 6). Humanized variants V1-V9 were tested for binding to BJAB cells, an immortalized B cell line which endogenously expresses HLA-DR2, at a single concentration of 10 μg/mL (67 nM). The humanized variants were compared to the parental chimeric 44H10 antibody composed of the mouse 44H10 antibody variable regions joined to the human IgG1 constant region. Positive binders were defined as those that had >4-fold increase in mean fluorescence intensity (MFI) over background. Results from this binding screened showed that none of the variants V1-V9 exhibited significant binding to the BJAB cell line (FIG. 10).

TABLE 5
List of VH and VL sequences used for
generation of humanized variants V1-V9.

| Construct | Sequence |
|---|---|
| VH1 | QVQLQESGPGLVKPSETLSLTCTVSGFSLTSYGW |
| | SWIRQPPGKGLEWIGYIWAGGSINYNPSLKSRVT |
| | ISVDTSKNQFSLKLSSVTAADTAVYYCARAYGDY |
| | VHYAMDYWGQGTLVTVSS (SEQ ID NO. 42) |
| VH2 | QVTLKESGPVLVKPTETLTLTCTVSGFSLTSYGV |
| | SWIRQPPGKALEWLAHIWAGGSISYSTSLKSRLT |
| | ISKDTSKSQVVLTMTNMDPVDTATYYCARAYGDY |
| | VHYAMDYWGQGTLVTVSS (SEQ ID NO. 43) |
| VH3 | QVQLQESGPGLVKPSGTLSLTCAVSGFSLTSYGW |
| | SWVRQPPGKGLEWIGEIWAGGSINYNPSLKSRVT |
| | ISVDKSKNQFSLKLSSVTAADTAVYYCARAYGDY |
| | VHYAMDYWGQGTLVTVSS (SEQ ID NO. 44) |
| VL1 | DIQMTQSPSSLSASVGDRVTITCRASQEISGYLA |
| | WFQQKPGKAPKSLIYAASSLQSGVPSRFSGSGSG |
| | TDFTLTISSLQPEDFATYYCLQYTNYPLTFGQGT |
| | KLEIK (SEQ ID NO. 45) |
| VL2 | DIQMTQSPSSLSASVGDRVTITCRASQEISGYLN |
| | WYQQKPGKAPKLLIYAASSLQSGVPSRFSGSGSG |
| | TDFTLTISSLQPEDFATYYCLQYTNYPLTFGQGT |
| | KLEIK (SEQ ID NO. 46) |
| VL3 | AIQMTQSPSSLSASVGDRVTITCRASQEISGYLG |
| | WYQQKPGKAPKLLIYAASSLQSGVPSRFSGSGSG |
| | TDFTLTISSLQPEDFATYYCLQYTNYPLTFGQGT |
| | KLEIK (SEQ ID NO. 47) |

TABLE 6
List of constructs used to generate humanized variants V1-V9.

| Humanized variant | VH Construct # | VL Construct # |
|---|---|---|
| V1 | 1 | 1 |
| V2 | 1 | 2 |
| V3 | 1 | 3 |
| V4 | 2 | 1 |
| V5 | 2 | 2 |
| V6 | 2 | 3 |
| V7 | 3 | 1 |
| V8 | 3 | 2 |
| V9 | 3 | 3 |

It is recognized that multiple CDR definition schemes exist for CDR grafting in humanization and that the lengths of the CDRs based on these definitions vary³. The approach used in generating humanized variants V1-V8 utilized the IMGT definition scheme, since it requires the shortest amount of foreign content to be grafted during humanization. However, differences in binding can be observed by changing the CDR scheme used for grafting during humanization⁴. Therefore, a potential explanation for the observed absence of binding to HLA-DR for the V1-V8 humanized variants is that critical contact residues outside of the IMGT-defined grafted CDR regions were mutated during the humanization process.

Based on this hypothesis, the humanization of 44H10 was re-attempted using the KABAT CDR definition scheme, which results in the grafting of longer CDRs in the CDRH2, CDRL1 and CDRL2. Given the long CDRH2 length using the KABAT definition, in certain cases, only select residues of the CDRH2 were replaced in order to limit the amount of murine content grafted. An additional 9 humanized variants, denoted as V10-V18, were generated from 3 new VH and VL constructs (Table 7 and Table 8). Humanized variants V10-V18 were tested for binding to BJAB cells at a single concentration of 10 μg/ml (67 nM). Results from this binding screen showed that only one humanized variant, V17, exhibited binding to BJAB cells using a >4-fold signal over background cut-off.

TABLE 7
List of VH and VL sequences used for
generation of humanized variants V10-V18.

| Construct | Sequence |
|---|---|
| VH4 | QVQLQESGPGLVKPSETLSLTCTVSGFSLTSYGV |
| | HWIRQPPGKGLEWIGYIWAGGSINYNPSLKSRVT |
| | ISVDTSKNQFSLKLSSVTAADTAVYYCARAYGDY |
| | VHYAMDYWGQGTLVTVSS (SEQ ID NO. 48) |
| VH5 | QVQLQESGPGLVKPSETLSLTCTVSGFSLTSYGV |
| | HWIRQPPGKGLEWIGVIWAGGSINYNSALMSRVT |
| | ISVDTSKNQFSLKLSSVTAADTAVYYCARAYGDY |
| | VHYAMDYWGQGTLVTVSS (SEQ ID NO. 49) |
| VH6 | QVTLKESGPVLVKPTETLTLTCTVSGFSLTSYGV |
| | HWIRQPPGKALEWLAVIWAGGSISYSTSLMSRLT |
| | ISKDTSKSQVVLTMTNMDPVDTATYYCARAYGDY |
| | VHYAMDYWGQGTLVTVSS (SEQ ID NO. 33) |
| VL4 | DIQMTQSPSSLSASVGDRVTITCRASQEISGYLT |
| | WYQQKPGKAPKLLIYAASTLDSGVPSRFSGSGSG |
| | TDFTLTISSLQPEDFATYYCLQYTNYPLTFGQGT |
| | KLEIK (SEQ ID NO. 50) |
| VL5 | DIQMTQSPSSLSASVGDRVTITCRASQEISGYLT |
| | WLQQKPGKAPKLLIYAASTLDSGVPSRFSGSGSG |
| | TDFTLTISSLQPEDFATYYCLQYTNYPLTFGQGT |
| | KLEIK (SEQ ID NO. 51) |
| VL6 | DIQMTQSPSSLSASVGDRVTITCRASQEISGYLT |
| | WFQQKPGKAPKSLIYAASTLDSGVPSRFSGSGSG |
| | TDFTLTISSLQPEDFATYYCLQYTNYPLTFGQGT |
| | KLEIK (SEQ ID NO. 52) |

TABLE 8
List of constructs used to generate humanized variants V10-V18.

| Humanized variant | VH Construct # | VL Construct # |
|---|---|---|
| V10 | 4 | 4 |
| V11 | 4 | 5 |
| V12 | 4 | 6 |
| V13 | 5 | 4 |
| V14 | 5 | 5 |
| V15 | 5 | 6 |
| V16 | 6 | 4 |
| V17 | 6 | 5 |
| V18 | 6 | 6 |

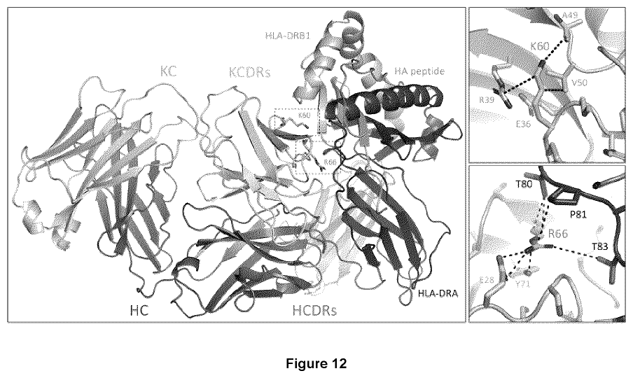

Despite these encouraging results, the binding of humanized variant V17 was significantly reduced compared to the parental 44H10 antibody. One hypothesis to explain this reduction in binding to HLA-DR was that critical contact residues in the framework regions of 44H10 were mutated during humanization. Although less common, a comprehensive analysis of published antibody: antigen crystal structures has shown that residues outside of the CDRs can contribute directly to binding interactions with antigen⁵. In an effort to identify if critical contact residues to HLA-DR exist within the framework regions of 44H10, we solved the crystal structure of the 44H10 Fab: HLA-DR complex (FIG. 12).

Surprisingly, this crystal structure revealed a disproportionate contribution of the Fab light chain to the interaction with HLA-DR (BSA=654.8 Å2), compared to its heavy chain counterpart (BSA=397.1 Å²). The angle of 44H10 Fab binding relative to the HLA-DR molecule positioned its light chain at the center of the interaction, enabling it to contact both the HLA-DR α and β chains. Conversely, the more distant positioning of the heavy chain only enabled it to contact the HLA-DR a chain. Accordingly, the 44H10 Fab angle of approach positioned its light chain framework region-region between the CDRL2 and CDRL3 (FW3)—in proximity to both HLA-DR chains, revealing unforeseen interactions between key framework residues and HLA-DR, namely K60 and R66.

The K60 side chain was found to participate in Van der Waals (VDW) interactions with multiple β chain residues, with a total BSA of ˜78 Å². R66 was found to be positioned in close proximity to the CDRL1 and CDRL2, possibly playing a role in loop stabilization, and with its side chain contacting multiple residues in the HLA-DR α chain (80-83) and burying a total BSA of ˜66 Å².

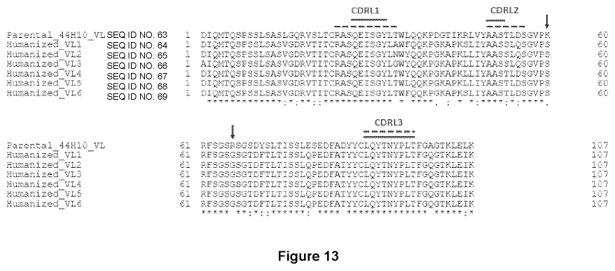

Based on the molecular insights gained from the 44H10 Fab: HLA-DR co-crystal structure, humanized constructs VL1-VL6 were analysed for their presence of the critical contact residues K60 and R66 in the FW3 of the 44H10 VL domain to determine if the observed loss or reduction in binding may be attributed to these residues being mutated during humanization. Sequence analysis of the variable regions showed that in all humanized VL1-6 constructs, K60 was mutated to a serine and R66 to a glycine (FIG. 13).

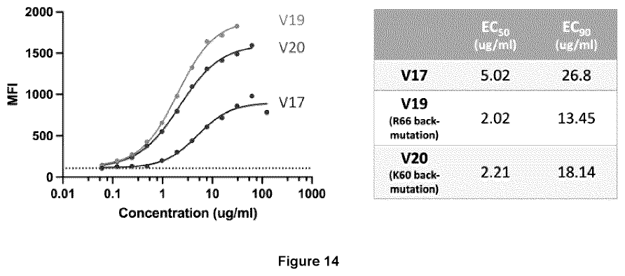

Therefore, using a structure-guided approach, the humanized VL5 construct of V17 was selected as a template to insert either the K60 or R66 back-mutations, based on this candidate's improved binding relative to the other humanized variants. This resulted in two additional VL constructs being synthesized that incorporated either the single K60 or R66 back-mutations (Table 9). These constructs were paired with the VH6 of humanized variant V17 to generate humanized variants 19 and 20 (Table 10). Variants V17, V19 and V20 were tested for binding on BJAB cells in a dose response (FIG. 14). Consistent with the structural data generated in FIG. 12, the insertion of either the R66 or K60 back-mutations in V19 and V20 significantly increased binding to BJAB cells compared to V17, as demonstrated by both increases in MFI signals and improvements in potency as measured by EC_{50/90}.

TABLE 9
List of VL sequences used for
generation of humanized variants V19-21.

| Construct | Sequence |
|---|---|
| VL7 | DIQMTQSPSSLSASVGDRVTITCRASQEISGYLT |
| | WLQQKPGKAPKLLIYAASTLDSGVPSRFSGSRSG |
| | TDFTLTISSLQPEDFATYYCLQYTNYPLTFGQGT |
| | KLEIK (SEQ ID NO. 34) |
| VL8 | DIQMTQSPSSLSASVGDRVTITCRASQEISGYLT |
| | WLQQKPGKAPKLLIYAASTLDSGVPKRFSGSGSG |
| | TDFTLTISSLQPEDFATYYCLQYTNYPLTFGQGT |
| | KLEIK (SEQ ID NO. 35) |
| VL9 | DIQMTQSPSSLSASVGDRVTITCRASQEISGYLT |
| | WLQQKPGKAPKLLIYAASTLDSGVPKRFSGSRSG |
| | TDFTLTISSLQPEDFATYYCLQYTNYPLTFGQGT |
| | KLEIK (SEQ ID NO. 36) |

TABLE 10
List of constructs used to generate humanized variants V19-V21.

| Humanized variant | VH Construct # | VL Construct # |
|---|---|---|
| V19 | 6 | 7 |
| V20 | 6 | 8 |
| V21 | 6 | 9 |

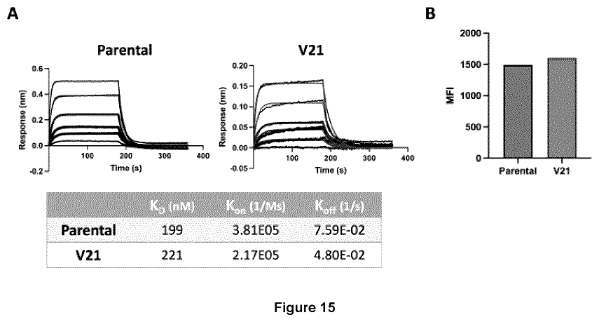

Residues K60 and R66 were subsequently both reverted in VL5 to generate VL9 (Table 9). VL9 was similarly paired with VH6 to generate V21 (Table 10) in order to determine if reverting both K60 and R66 rescued binding of the humanized variants to HLA-DR. FIG. 15 shows that the binding of V21 to HLA-DR was completely restored to levels comparable to the parental 44H10 mAb by both affinity measurements using biolayer interferometry (A), as well as flow cytometry experiments using BJAB cells (B).

### REFERENCES

1. Quackenbush, E. J. & Letarte, M. Identification of several cell surface proteins of non-T, non-B acute lymphoblastic leukemia by using monoclonal antibodies. J. Immunol. 134, 1276-85 (1985).
2. Menezes, J., Leibold, W., Klein, G. & Clements, G. Establishment and characterization of an 2. Epstein-Barr virus (EBC)-negative lymphoblastoid B cell line (BJA-B) from an exceptional, EBV-genome-negative African Burkitt's lymphoma. Biomedicine 22, 276-284 (1975).
3. Dondelinger, M. et al. Understanding the significance and implications of antibody numbering and antigen-binding surface/residue definition. Front. Immunol. 9, 2278 (2018).
4. MacCallum, R. M., Martin, A. C. R. & Thornton, J. M. Antibody-antigen Interactions: Contact Analysis and Binding Site Topography. J. Mol. Biol. 262, 732-745 (1996).
5. Kunik, V., Peters, B. & Ofran, Y. Structural Consensus among Antibodies Defines the Antigen Binding Site. PLOS Comput. Biol. 8, e1002388 (2012).
6. Scally, S. W. et al. A molecular basis for the association of the HLA-DRB1 locus, citrullination, and rheumatoid arthritis. J. Exp. Med. 210, 2569 (2013).
7. Kabsch, W. & IUCr. XDS. urn:issn:0907-4449 66, 125-132 (2010).
8. McCoy, A. J. et al. Phaser crystallographic software. urn:issn:0021-8898 40, 658-674 (2007).
9. Adams, P. D. et al. PHENIX: a comprehensive Python-based system for macromolecular structure solution. urn:issn:0907-4449 66, 213-221 (2010).
10. Emsley, P., Lohkamp, B., Scott, W. G. & Cowtan, K. Features and development of Coot. urn:issn:0907-4449 66, 486-501 (2010).
11. Morin, A. et al. Collaboration gets the most out of software. Elife 2013, (2013).

### Example 11

In this study, it was determined from analysis of the chimeric 44H10 Fab-HLA-DR co-crystal structure that the HLA-DR α chain contributes 2 times more BSA than the β chain and that all but 3 contacted HLA-DR residues are conserved across alleles. Sequences of known HLA-DRA and HLA-DRB1 alleles were aligned using the IPD-IMGT/HLA database sequence alignment tool, and interactions were analyzed using the PDBePisa server. For binding studies, HLA-DR variants with substitutions in the three identified peripheral residues displaying slight sequence diversity (namely α-W168, β-F31, and β-H33) were expressed and purified as described above.

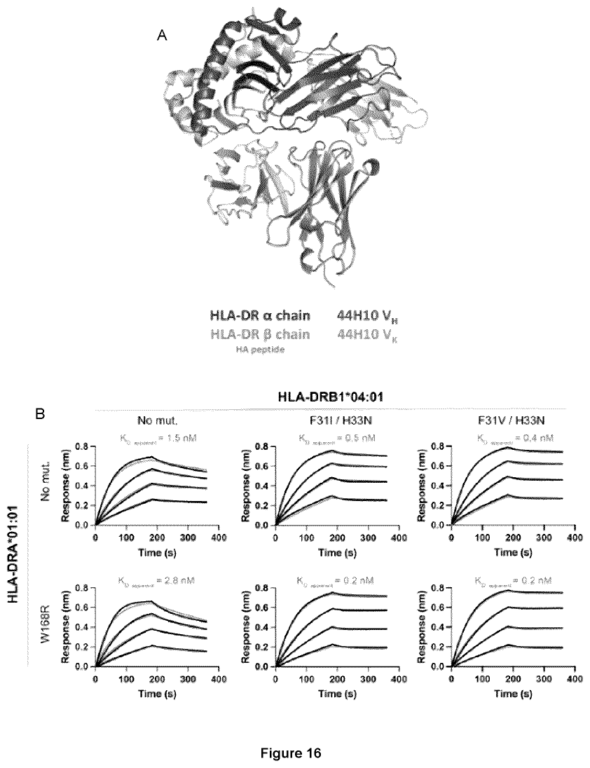

Real-time analysis of binding kinetics was measured using the Octet RED96 BLI system (Sartorius). Baseline, association, and dissociation steps were conducted at 25° C. for 180 s in kinetics buffer (PBS, pH 7.4, 0.01% BSA, 0.002% Tween). Recombinant HLA-DR was loaded onto Penta-His Biosensors (ForteBio) at 10 μg/mL until a threshold response of 0.7 nm. Association events were measured by dipping loaded biosensors into wells containing a two-fold serial dilution of chimeric 44H10 IgG at a 250 nM starting concentration. Dissociation was measured by transfer of biosensors back into buffer-containing wells. Biosensors were regenerated in 10 mM glycine, pH 1.5. Kinetics data were analyzed using the FortéBio Octet Data Analysis software 9.0.0.6, and curves were fitted to a 1:1 binding model for calculation of K_{D}, Kₒₙ and K_{off}. Substitutions in the three variable residues peripheral to the 44H10 epitope did not impact 44H10 binding (FIGS. 16A and 16B).

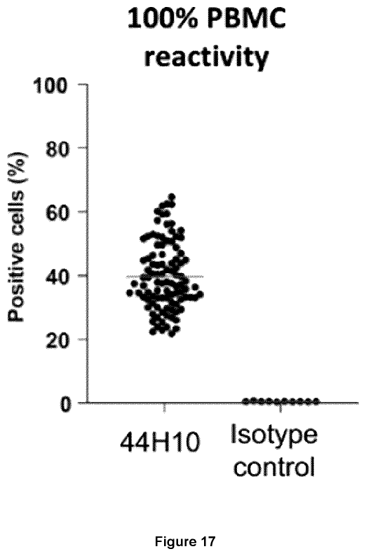

It was found that 44H10 was reactive to 100/100 donor PBMC samples (FIG. 17). Peripheral blood mononuclear cells (PBMC) cells from healthy human donors were isolated by density centrifugation using Ficoll-Hypaque solution (GE Healthcare). PBMC were resuspended in staining buffer (PBS, 2% FCS, 1 mM EDTA) and Fc receptors were blocked with Human TruStain FcX™ (Biolegend) according to the manufacturer's instructions. PBMCs were then stained for 1 h at 4° C. with 50 μL of 0.1 mg/mL AF488 pre-labeled c44H10 IgG. AF488-conjugated Human IgG1 was used as isotype control. Samples were acquired on a Sony SP6800 Spectral Analyzer and processed using the FlowJo™ Software.

### Example 12

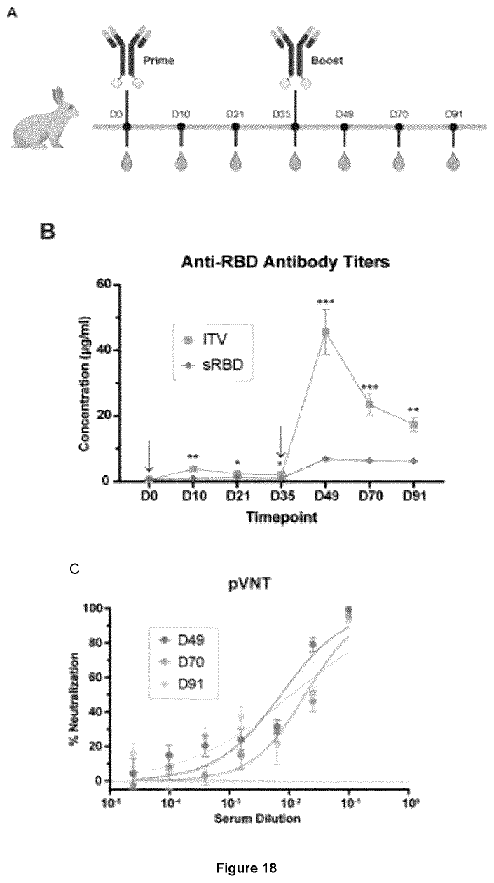

In this study, it is shown that immunization with the immunotargeting vaccine (chimeric 44H10) induced robust anti-RBD IgG titers and neutralization titers, particularly after a booster dose at day 35 post-priming (FIGS. 18A, 18B, and 18C). Female New Zealand white rabbits were immunized at day 0 (DO) via SQ injection with unadjuvanted RBD or ITV, followed by a boost at D35. For serum preparation, blood was collected into red top vacutainer tubes and incubated at 3-4 h at room temperature to allow for clotting. The tubes were centrifuged at 4° C. at 3000 rpm for 20 min, and supernatants were poured off into appropriate tubes and stored at −20° C.

For measurement of RBD-specific antibody titers, Immulon 4 HBX ELISA plates (Thermo Scientific) were coated overnight at 4° C. with 100 ng/well SARS-CoV-2 spike RBD (produced in-house). All subsequent steps were conducted at room temperature. Plates were washed three times with PBS-T (PBS, 0.1% Tween), then incubated with blocking buffer (PBS-T, 3% non-fat milk) for 1 h. The blocking solution was discarded and 100 μL of rabbit sera pre-diluted in diluent buffer (PBS-T, 1% milk) and standard (rabbit anti-SARS-CoV-2 spike RBD polyclonal antibody, Cedarlane) were added to the ELISA plates. After a 2 h incubation, plates were washed three times with PBS-T and incubated with Goat Anti-Rabbit IgG H&L (HRP) secondary antibody (1:10,000, Abcam) for 1 h. Plates were once more washed three times, then developed using a TMB Substrate Reagent Set (BD) following manufacturer instructions; reactions were stopped at 5 min by the addition of 2 N HCl. Absorbance readings at 450 nm were acquired using a Synergy Neo2 Multi-Mode Assay Microplate Reader (Biotek Instruments). Data were plotted in Prism v9.3.1 (GraphPad) and antibody concentration was extrapolated from absorbance using four-parameter logistic (4PL) regression of log-transformed values.

For measurement of neutralizing antibody responses by pseudovirus neutralization assay, 293T cells were co-transfected with a lentiviral backbone encoding the luciferase reporter gene (BEI NR52516), a plasmid expressing the SARS-CoV-2 Spike (BEI NR52310) and plasmids encoding the HIV structural and regulatory proteins Tat (BEI NR52518), Gag-pol (BEI NR52517) and Rev (BEI NR52519). Co-transfection of the five plasmids was performed using BioT reagent (Bioland Scientifics) following manufacturer instructions. 24 h post-transfection at 37° C., the media was supplemented with 5 mM sodium butyrate (NaB) and the cells were incubated for an additional 24 h at 30° C. prior to pseudovirus (PsV) harvesting. PsV were harvested, filtered through 0.45 μm sterile filters, and concentrated using 100 K Amicon filters (Millipore Sigma). Neutralization assays were performed using 293T-ACE2 cells (BEI NR52511). Rabbit sera were inactivated for 30 min at 56° C. Serial dilutions of the inactivated sera were incubated for 1 h at 37° C. with SARS-CoV-2 PsV and subsequently added to 293T-ACE2 cells (BEI NR52511) seeded in Poly-L-lysine (Sigma-Aldrich) coated plates 24 h prior to the experiment. A final concentration of 5 μg/ml polybrene (Sigma-Aldrich) was added to the PsV-sera mixtures. After 48 h of incubation at 37° C., neutralization was monitored by adding 50 ul of Britelite plus reagent (PerkinElmer) to 50 ul of cells for 2 min. Supernatants were transferred to a 96-well white plate (Sigma-Aldrich) to measure luminescence in relative light units (RLUs) using a Synergy Neo2 Multi-Mode Assay Microplate Reader (Biotek Instruments). Absorbance data were converted to % inhibition using the same formula as used in the sVNT. Data were fitted using 4PL regression constrained at top=100% and bottom=0% in GraphPad Prism 9.3.1 for determination of neutralization titers.

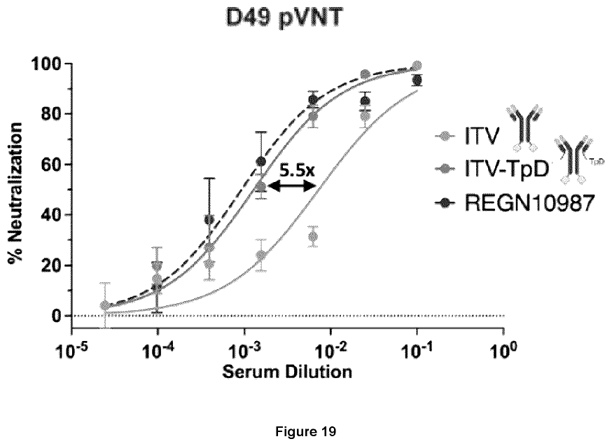

It was further shown that the addition of a TpD TCE enhanced peak (D49) sera neutralization potency more than 5-fold (ED₅₀=0.0013) as measured by pVNT, with peak antibody responses roughly equivalent to 100 μg/ml of the highly neutralizing therapeutic mAb REGN 10987 (FIG. 19). Pre-immunization with T/D resulted in an additional >3× boost in neutralization titers, providing an opportunity to leverage pre-existing immunity with vaccination with the immunotargeting mAb.

### Example 13

Ferrets were immunized intramuscularly with 50 μg of unadjuvanted ITV-TpD or an RBD-equimolar dose of Alum-adjuvanted RBD on DO and D35. Unvaccinated ferrets were immunized with an equal volume of sterile PBS. Each group of six animals consisted of three males and three females. SARS-CoV-2 (hCoV-19/Canada/ON-VIDO-01/2020) (VIDO) was grown and maintained in VeroE6 cells at CFIA-NCFAD Level 3 (Zoonotic). Ferrets were challenged on D49 with 10⁶ pfu SARS-CoV-2 (Wuhan-Hu-1) intranasally (50 μL per nostril, 100 μL total). Blood samples were collected in BD serum-separator microtainer tubes (BD 365967) except on D14 post-challenge, where blood was collected in 5 mL BD vacutainer serum-separator tubes (BD 367989) upon euthanasia. Serum separated from blood and nasal washes performed using 1 mL PBS were stored in 2 mL screw cap micro tubes (Sarstedt) at −80° C. until use.

For the ELISA measurement of anti-RBD titers elicited in ferrets, 96-well flat bottom plates (Nunc) (Thermo Scientific) were coated with 50 ng/well of RBD in 0.05 M carbonate-bicarbonate buffer (Sigma-Aldrich) overnight at 4° C. Plates were washed 5 times with 0.01 M PBS-T and blocked with 1× Casein Blocking Buffer (Sigma-Aldrich) for 1 h shaking at 37° C. Plates were washed and dilutions of serum samples were prepared in casein blocking buffer and incubated for 1 h shaking at 37° C. Plates were washed and incubated with goat anti-ferret IgG-HRP (1:10,000, Abcam) for 1 h shaking at 37° C. Plates were washed and developed with TMB (ThermoFisher Scientific) following manufacturer instructions; reactions were stopped by the addition of Stop Solution 0.16 M sulphuric acid (ThermoFisher Scientific). Absorbance of the plates was read at 450 nm.

For the plaque reduction neutralization titer (PRNT) assay, serum samples were heat-inactivated at 56° C. for 30 min. Two-fold serial dilutions of inactivated sera were incubated with 100 pfu of SARS-CoV-2 virus for 1 h at 37° C. Each virus-serum mixture was then added to wells of >90% confluent Vero E6 cells in a 48-well format, incubated for 1 h at 37° C. in 5% CO₂, then overlaid with 500 UL of 2% carboxymethyl cellulose (Sigma) in supplemented DMEM (Corning) per well. Plates were incubated at 37° C. for 72 h, fixed with 10% buffered formalin and stained with 0.5% crystal violet. Serum dilutions resulting in >70% reduction of plaque counts compared to virus-only controls were considered positive for virus neutralization.

For the RT-qPCR measurement of viral titers in nasal washes, total RNA extraction from nasal washes was conducted using the MagMAX CORE Nucleic Acid Purification Kit (ThermoFisher) on the Thermo Scientific Kingfisher benchtop automated extraction instrument, using TriPure Isolation reagent (Sigma Aldrich) in a 1:9 v/v ratio instead of the kit-supplied Lysis Solution. 650 μL inactivated sample, 30 μL binding beads and 350 μL binding buffer spiked with Armoured RNA-Enterovirus (ARM-ENTERO, Asuragen) were then used for RNA extraction in 96 deep-well plates. Extracted RNA was recovered in 30 μL elution buffer. The spiked enteroviral armoured RNA was used as an exogenous RNA extraction control. RNA extracted from nasal washes was tested for the presence of SARS-CoV-2 RNA by an E gene RT-qPCR that detects a broad range of human and bat coronaviruses⁸¹. For the detection of SARS-CoV-2 RNA by RT-qPCR, 4× TaqMan Fast Virus one step RT-PCR kit (LifeTech) was used according to manufacturer's recommendations. For each RT-qPCR reaction, 0.4 μM of E gene forward and reverse primers, 0.2 μM of ARM-ENTERO forward and reverse primers and 0.2 μM of both probes were used. RT-qPCR runs were performed using a 7500 Fast Real-Time PCR system (Applied Biosystems) using the following cycle conditions: 50° C. for 5 min, 95° C. for 20 s, then 40 cycles of 95° C. for 3 s followed by 60° C. for 30 s. RT-qPCR semi-quantitative results were calculated based on a gBlock (Integrated DNA Technologies) standard curve for SARS-CoV-2 E gene.

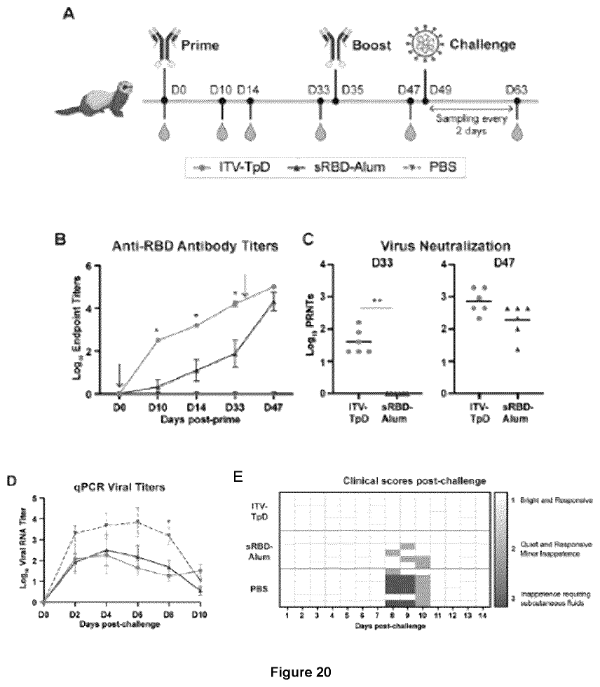

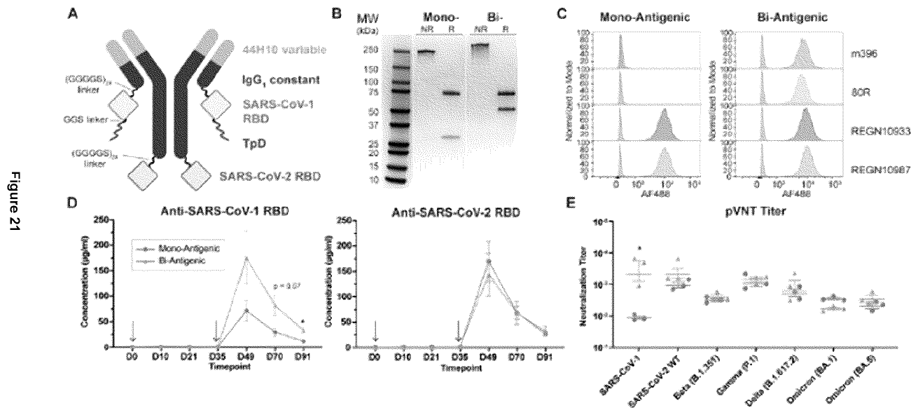

In this study, it is shown that adjuvant-free immunization with the immunotargeting vaccine (chimeric 44H10 with TpD epitope tag) induced robust anti-RBD IgG titers in ferrets (FIGS. 20A and 20B) and neutralized virus in vivo following challenge (FIGS. 20C and 20D), resulting in improved clinical scores post-challenge (FIG. 20E).

### Example 14

In this study, it is shown how the modular immunotargeting vaccine design allows for inclusion of multiple RBDs to achieve broad sarbecovirus neutralization. To produce a bi-antigenic immunotargeting vaccine, the SARS-CoV-2 and SARS-CoV-1 spike protein RBDs were respectively fused to the c44H10 IgG heavy and light chains, as shown in FIG. 21A. With consideration to our results on the importance of TCEs for ITV immunogenicity, TpD was also fused to the C terminus of the SARS-CoV-1 RBD on the ITV light chain. This molecule was expressed, purified and characterized by SDS-PAGE and flow cytometry as previously described. FIGS. 21B, 21C, and 21D show results of characterizing the mono-antigenic design and the bi-antigenic design. Following immunization of rabbits with unadjuvanted mono- and bi-antigenic immunotargeting vaccine, antibody responses were assessed by ELISA and pseudovirus neutralization assay as previously described. FIG. 21E shows the virus neutralizing ability of the modular immunotargeting vaccine against various sarbecoviruses, demonstrating enhanced SARS-CoV-1 neutralizing potency in rabbits immunized with bi-antigenic vaccine, and comparable neutralization against wild-type and variant SARS-CoV-2 pseudoviruses.

### Example 15

V14, V17 and V21 Fabs for crystallization were transfected as described above and purified by KappaSelect affinity chromatography, followed by MonoS ion exchange chromatography using 20 mM NaOAc, pH 5.6+1M KCl, and finally size exclusion chromatography on a Superdex 200 Increase 10/300 GL in TBS, pH 8 (all from Cytiva). Fabs were concentrated to 9.7-10.4 mg/mL and protein crystals were grown in Top96, MCSG1 and MCSG2 crystallization screens. Protein crystal diffraction data were collected at the 23-ID-B and 23-ID-D beamlines at the Argonne National Laboratory Advanced Photon Source. Structures were solved as described above for the chimeric 44H10 Fab-HLA-DR complex. Interactions were analyzed using the PDBePisa server and hbplus software.

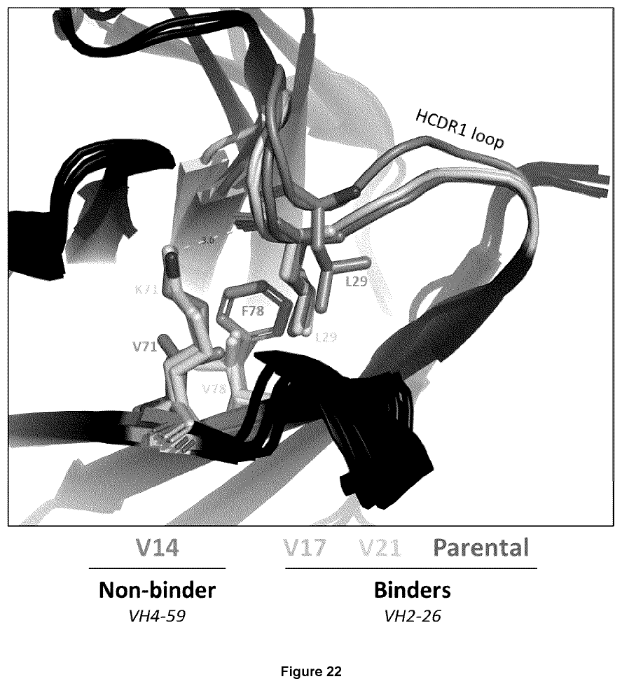

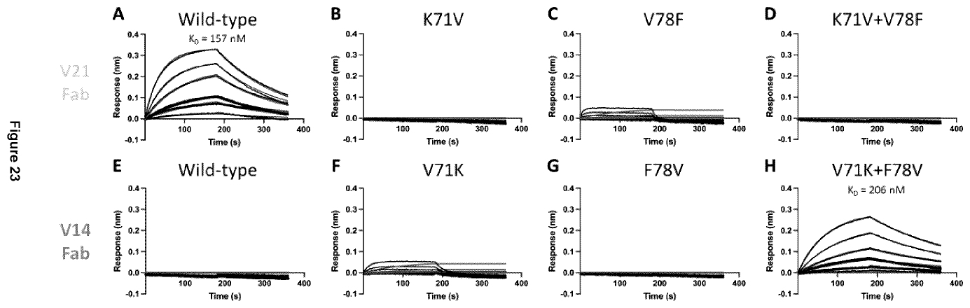

In this study, analysis of the structures of V14, V17, V21 and parental 44H10 mAb showed that VH residues at framework positions 71 and 78 impact HCDR1 conformation (FIG. 22). In the parental 44H10 and VH2-26 mAbs (V21, V17), the K71 side chain H-bonds with the L29 main chain, stabilizing the HCDR1 loop. V71 in VH4-59 mAbs V14 is not able to mediate this interaction. Small residues at position 78 (e.g. V78 in parental 44H10 and VH2-26 mAbs) allow the interaction between K71 and L29, but bulky residues (e.g. F78 in VH4-59 mAbs) hinder this interaction. Significantly increased flexibility in the VH4-59 HCDR1 modulates binding to HLA-DR through its downstream effects on the antibody paratope.

Mutagenesis and binding experiments by biolayer interferometry (BLI) were subsequently performed to confirm these structural observations. Mutating K71 to a V in V21 (binder) completely knocked out binding to recombinant HLA-DR (FIGS. 22A and 22B). Conversely, mutating V71 to a K in V14 (non-binder) restored some HLA-DR binding (FIGS. 22E and 22F). Steric hindrance caused by the V78F mutation in V21 on the K71-L29 interaction significantly reduced binding (FIGS. 22A and 22C). Individually, the removal of this steric hindrance in V14 through the F78V mutation was unable to restore binding (FIGS. 22E and 22G). Together, the V71K and F78V mutations restore V14 binding at an affinity almost equivalent to V21 (FIGS. 22E and 22H). Pairing of the V14 HC with V71K+F78V mutations to a LC with G66R+S60K mutations likely results in a humanized candidate that surpasses V21 binding to HLA-DR.

What is claimed is:

1. A humanized anti-class II MHC antibody, wherein the humanized antibody binds to class II MHC with similar or increased affinity and/or specificity as compared with a non-humanized anti-MHC class II antibody that specifically binds to a shared epitope on most or all HLA-DR molecules.

2. The antibody of claim 1, wherein the non-humanized anti-MHC class II antibody is based on 44H10.

3. The antibody of claim 1 or 2, wherein the non-humanized MHC class II antibody is a human-mouse chimeric anti-class II MHC antibody based on 44H10.

4. The antibody of any one of claims 1 to 3, wherein the non-humanized MHC class II antibody comprises an amino acid sequence having at least 70% identity to SEQ ID NO. 31, 32, or a fragment thereof.

5. The antibody of claim 1, wherein the antibody is an anti-HLA-DR antibody.

6. The antibody of claim 2, wherein the antibody is broadly reactive with most or all HLA-DR molecules.

7. The antibody of any one of claims 1 to 3, wherein the antibody is an IgG, scFv, Fab′, Fab, F(ab′)₂, or scFab.

8. The antibody of claim 4, wherein the antibody is an IgG.

9. The antibody of any one of claims 1 to 8, wherein the antibody is a monoclonal antibody.

10. The antibody of any one of claims 1 to 9, wherein the antibody has a 44H10 specificity.

11. The antibody of any one of claims 1 to 10, wherein the antibody comprises a VH construct comprising an amino acid sequence having at least 70% identity to SEQ ID NO. 33 or a fragment thereof.

12. The antibody of any one of claims 1 to 11, wherein the VH construct comprises a mutation that increases binding to HLA-DR.

13. The antibody of claim 12, wherein the mutation is at position 71 and/or 78.

14. The antibody of claim 12 or 13, wherein the mutation results in a K at position 71 and/or a V at position 78.

15. The antibody of any one of claims 1 to 14, wherein the antibody comprises a VL construct comprising an amino acid sequence having at least 70% identity to SEQ ID NO. 34, 35, 36, or a fragment thereof.

16. The antibody of any one of claims 1 to 15, wherein the VL construct comprises a mutation that increase binding to HLA-DR.

17. The antibody of claim 16, wherein the mutation is at position 60 and/or 66.

18. The antibody of claim 16 or 17, wherein the mutation results in a K at position 60 and/or an R at position 66.

19. The antibody of any one of claims 1 to 18, conjugated to another molecule.

20. The antibody of claim 13 wherein the molecule comprises singularly or in combination a polypeptide or protein, a carbohydrate, a polynucleotide, a small molecule, or a lipid.

21. The antibody of claim 20, wherein the polypeptide comprises an antigen.

22. The antibody of claim 21, wherein the antigen is from an infectious agent.

23. The antibody of claim 22, wherein the infectious agent is a coronavirus.

24. The antibody of claim 23, wherein the coronavirus is SARS-CoV-1, SARS-CoV-2, or MERS.

25. The antibody of claim 23 or 24, wherein the coronavirus antigen is a spike protein antigen or a nucleocapsid antigen.

26. The antibody of claim 25, wherein the coronavirus antigen is an S1 antigen or an S2 antigen.

27. The antibody of claim 26, wherein the coronavirus antigen is an RBD antigen.

28. The antibody of any one of claims 24 to 27, wherein the coronavirus antigen comprises a polypeptide having at least 70% identity to SEQ ID NO. 37, 38, 39, 40, 41, or a fragment thereof.

29. The antibody of any one of claims 19 to 28, wherein the molecule is conjugated to a heavy chain of the antibody.

30. The antibody of claim 29, wherein the molecule is conjugated at the C-terminus of the heavy chain.

31. The antibody of any one of claims 19 to 30, wherein the molecule is conjugated to a light chain of the antibody.

32. The antibody of claim 31, wherein the molecule is conjugated at the C-terminus of the light chain.

33. The antibody of any one of claims 29 to 32, wherein the antibody comprises two heavy chains and/or two light chains and a plurality of molecules, either the same or different, each conjugated to a different heavy chain and/or light chain.

34. The antibody of claim 33, comprising two or more molecules, each conjugated to a respective heavy chain or light chain, wherein the molecules are independently the same or different.

35. The antibody of claim 34, comprising three or four molecules, each conjugated to a respective heavy chain or light chain, wherein the molecules are independently the same or different.

36. The antibody of claim 35, comprising four molecules, one at each heavy and light chain.

37. The antibody of claim 36, wherein the molecules at each heavy chain are the same and the molecules at each light chain are the same, and wherein the molecules at each heavy chain are different from the molecules at each light chain.

38. The antibody of claim 33, comprising two molecules, each conjugated to a respective heavy chain.

39. The antibody of any one of claims 19 to 38, further comprising a linker between the antibody and the molecule.

40. The antibody of claim 39, wherein the linker is a GS repeat linker, such as a GGSx2 linker or GGGGSx2 linker.

41. The antibody of any one of claims 19 to 40, wherein the molecule is a universal T-helper determinant.

42. The antibody of claim 41, wherein the universal T-helper determinant comprises PADRE and/or TpD.

43. The antibody of claim 41 or 42, wherein the universal T-helper determinant is conjugated to a heavy chain of the antibody.

44. The antibody of claim 43, wherein the universal T-helper determinant is conjugated at the C-terminus of the heavy chain.

45. The antibody of any one of claims 41 to 44, wherein the universal T-helper determinant is conjugated to a light chain of the antibody.

46. The antibody of claim 45, wherein the universal T-helper determinant is conjugated at the C-terminus of the light chain.

47. The antibody of any one of claims 43 to 46, wherein the antibody comprises two heavy chains and/or two light chains and comprising a plurality of universal T-helper determinants, either the same or different, each conjugated to a different heavy chain and/or light chain.

48. The antibody of claim 47, comprising two universal T-helper determinants, each conjugated to a respective heavy chain or light chain.

49. The antibody of claim 48, comprising two universal T-helper determinants, each conjugated to a respective light chain.

50. The antibody of any one of claims 41 to 49, further comprising a linker between the antibody and the universal T-helper determinant.

51. The antibody of claim 50, wherein the linker is a GS repeat linker, such as a GGSx2 linker or GGGGSx2 linker.

52. The antibody of any one of claims 41 to 51 for use in combination with a vaccine against tetanus and/or diphtheria toxoids.

53. The antibody of any one of claims 1 to 52 comprising a polypeptide sequence having at least 70% sequence identity to any one or more of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

54. The antibody of claim 53, comprising at least one heavy chain and at least one light chain of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, in any combination.

55. The antibody of claim 54, comprising two heavy chains and two light chains of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, in any combination.

56. The antibody of claim 55, consisting of two heavy chains and two light chains of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, in any combination.

57. A polynucleotide encoding the antibody of any one of claims 1 to 56.

58. A vaccine comprising the polynucleotide of claim 57.

59. A vector comprising the polynucleotide of claim 57.

60. A host cell comprising the vector of claim 59.

61. A vaccine comprising the antibody of any one of claims 1 to 56.

62. The vaccine of claim 61, wherein the vaccine is free of an adjuvant.

63. A method of immunizing a subject against a disease or condition, the method comprising administering the vaccine of claim 58, 61, or 62 to the subject.

64. A method of treating and/or preventing a disease or condition in a subject, the method comprising administering the vaccine of claim 58, 61, or 62 to the subject.

65. The method of claim 63 or 64, further comprising administering a vaccine against tetanus and/or diphtheria toxoids to the subject.

66. The method of claim 65, wherein the vaccine against tetanus and/or diphtheria toxoids is administered prior to administering the vaccine against the disease or condition.

67. The method of claim 65, wherein the vaccine against tetanus and/or diphtheria toxoids is administered to the subject prior to the vaccine against the disease or condition, such as one or more days, weeks, months, or years prior to the vaccine against the disease or condition, such as about one month prior to the vaccine against the disease or condition.

68. The method of any one of claims 63 to 67, wherein the vaccine against the disease or condition is administered without an adjuvant.

69. The method of any one of claims 63 to 68, wherein the vaccine against the disease or condition is administered as a purified protein without an adjuvant.

70. Use of the vaccine of claim 58, 61, or 62 for immunizing a subject against a disease or condition.

71. Use of the vaccine of claim 58, 61, or 62 for treating and/or preventing a disease or condition.

72. The use of claim 70 or 71, further comprising use of a vaccine against tetanus and/or diphtheria toxoids.

73. The use of claim 72, wherein the vaccine against tetanus and/or diphtheria toxoids is for use prior to the vaccine against the disease or condition, such as one more days, weeks, months, or years prior to the vaccine against the disease or condition, such as about one month prior to the vaccine against the disease or condition.

74. The use of any one of claims 70 to 73, wherein the vaccine against the disease or condition is for use without an adjuvant.

75. The use of any one of claims 70 to 73, wherein the vaccine against the disease or condition is for use as a purified protein without an adjuvant.
